(19) United States
(12) Patent Application Publication
(10) Pub. No.: US 2024/0293343 A1
(43) Pub. Date: Sep. 5, 2024

(54) Benzylideneaminoguanidine derivatives as NR2B-selective NMDA receptor antagonists and their therapeutic applications

(71) Applicant: InFlectis BioScience, Nantes (FR)

(72) Inventors: Pierre MINIOU, NANTES (FR); Philippe GUEDAT, MAUVES-SUR-LOIRE (FR)

(21) Appl. No.: 18/109,031

(22) Filed: Feb. 13, 2023

(51) Int. Cl. A61K 31/155; A61P 25/02

(52) U.S. Cl. CPC A61K 31/155; A61P 25/02

(57) ABSTRACT

The present invention relates benzylidene aminoguanidine derivatives as NR2B-selective NMDA receptor antagonists and their therapeutic applications.

The present invention relates to compounds that have potential therapeutic applications by preventing or treating a disease, disorder, or medical condition mediated by NR2B-containing N-methyl-D-aspartate (NMDA) receptor activity.

### BACKGROUND OF THE INVENTION

N-methyl-D-aspartate receptors (NMDARs) are ionotropic glutamate receptors permeable to Ca²⁺, Na⁺ and K⁺. NMDARs are critical for physiological synaptic plasticity in the developing and mature CNS. NMDARs are multi-subunit complexes associating NR1, NR2 and rarely NR3 subunits. Most NMDARs are tetrameric complexes consisting of two NR1 subunits and two NR2 subunits; hexameric complexes containing NR1/NR2/NR3 have also been identified. NR1 is encoded by a single gene with at least eight different splice variants, and NR2 by four different genes NR2A (GRIN2A), NR2B (GRIN2B), NR2C (GRIN2C) and NR2D (GRIN2D); two NR3 genes originates NR3A (GRIN3A) and NR3B (GRIN3B) subunits. To be activated, NMDARs need to bind glutamate via NR2 subunit, glycine via NR1 subunit and release Mg²⁺ blockade by membrane depolarization. Glutamate binding to NR2 subunit determines the duration of channel opening and desensitization processes. NMDARs containing different NR2 subunits have different pharmacological and kinetic properties. While the NR1 subunit is expressed in virtually all neurons and at all developmental stages in the brain, NR2 subunit genes display different regional and developmental expression patterns. NR2A subunits are widely expressed in the adult mammalian brain, while NR2B expression is restricted to the cortex, hippocampus, striatum, amygdala, ventral nuclei of the thalamus, the olfactory bulb and the dorsal horn of the spinal cord, NR2C subunit is expressed in the cerebellum, and NR2D is expressed in the midbrain. Outside the central nervous system, NMDARs are also present in Schwann cells.

The NMDAR has been a major target for drug development in neurology because preclinical researches have provided substantial amount of evidence for its role in cellular and animal models of many neurological diseases. NMDAR are best known for their role in excitotoxicity, a pathological process during which excessive glutamate release causes overactivation of NMDARs which leads to a massive influx of extracellular Ca²⁺ into the cells, followed by an increase in intracellular Ca²⁺ concentration to pathological levels. Increased intracellular Ca²⁺ levels may further lead to a series of downstream neurotoxic cascades, resulting in the increased formation of reactive oxygen species (ROS) and activation of caspase-dependent and caspase-independent cell death, in which mitochondria play a key role. This process has been implicated in both acute ischemic stroke and traumatic brain injury. Glutamate excitotoxicity contributes also, at least partly, to neuronal loss in chronic neurodegenerative conditions, including Alzheimer's disease (AD), and other dementias, Parkinson's disease (PD), Huntington's disease (HD), amyotrophic lateral sclerosis (ALS) and possibly in multiple sclerosis (MS) and prion's disease. Overactivity of excitotoxicity pathways is also observed in epilepsy and neuropathic pain.

First-generation NMDAR antagonists developed in the 1980-1990s bind to agonist binding domains (i.e. glycine or glutamate binding sites) or to the pore channel. They displayed preclinical effectiveness in different indications (e.g. excitotoxic neurodegeneration, neuropathic pain, ischemia-induced neurodegeneration, depression . . . ), but most of compounds, except memantine were abandoned because of unacceptable side effects (e.g. hallucinations, memory and motor deficits . . . ) due of their broad spectrum and their lack of subunit specificity. Glycine binding site competitive antagonists showed little receptor subtype selectivity, as expected for compounds targeting a binding site located on NR1, a subunit present in all receptor subtypes. NMDAR pore blockers usually discriminate poorly between NMDAR subtypes, NMDAR pore channel blockers dizocilpine (MK-801) and phencyclidine (PCP), induced in healthy individuals, psychotic and negative symptoms, as well as cognitive impairment, that resemble those present in schizophrenia and exacerbated these symptoms in schizophrenic patients which prohibit their widespread use. MK-801 has been also shown to create brain lesions in laboratory rats. Channel blockers ketamine and dextromethorphan which antagonizes NMDAR by binding a site within the channel pore, have been reported to produce symptomatic relief in several neuropathies, however these agents induce unacceptable side effects at analgesic doses including hallucinations, dysphoria and disturbances of cognitive and motor function. Memantine is the only NMDAR pore channel blocker approved compound for use in Alzheimer disease; its uncompetitive, low-affinity mechanism of action allows the blocking of excessive NMDAR activations produced by glutamate, while allowing normal activation of the NMDAR channel.

Subunit-selective NMDAR antagonists appeared to have a much-improved side effect profile compared with broad-spectrum antagonists. Thus, NR2B-selective antagonists have been the focus of intense study and development in the past years because of NR2B containing receptors tissue and sub-cellular localizations and their contributions to pathological processes linked to overexcitation of glutamatergic pathways. For example, in the adult spinal cord, NR2B expression is restricted to lamina 2 of the dorsal horn, a region that receives primary sensory afferents from nociceptors and thermoreceptors. This restricted localization of NR2B-containing receptors in this region could explain, in part, why NR2B-selective antagonists such as Ifenprodil, and its related structures (i.e. traxoprodil/CP101,606 and Ro25-6981) have analgesic effects. Thus, the therapeutic potential of NR2B-selective antagonists is well established (Mony et al. British J Pharmacol 2009; 157:1301-1317; Chazot P Current Medicinal Chemistry, 2004, 11, 389-396 389).

However, NR2B-selective antagonists have not yet been developed into approved drugs. Ifenprodil, the most promising NR2B negative allosteric modulator displayed a poor oral bioavailability and limitations due to its inhibition of GIRK channels, and its interaction with alpha1 adrenergic, serotonin, and sigma receptors. Traxoprodil development to treat chronic pain, PD, major depression has been halted, despite initially promising results, because of significant dissociative side effects. Although well tolerated, Rislenemdaz (also known as CERC-301 and MK-0657) did not provide clinically meaningful improvement in motor function in patients with moderate Parkinson's disease. In 2011, a phase II clinical trial for major depressive disorder of EVT-101 was terminated early due to a clinical hold issued bythe FDA (NCT01128452). In 2021, MIJ821 is the only NR2B-selective antagonist under evaluation in a phase II clinical trial for treatment-resistant depression (NCT03756129).

Thus, there continues to be a need for novel NMDA antagonists that target the NR2B receptor.

Some benzylideneguanidine derivatives of formula (I) are known from the literature. The compound 2-(2,6-dichlorobenzylidene)hydrazinecarboximidamide, also referred to as guanabenz, is an alpha adrenergic receptor agonist of the alpha-2 type that has been marketed as an antihypertensive drug.

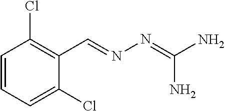 [SMILES: NC(N)=N/N=C/c1c(Cl)cccc1Cl]

Its therapeutic potential in several other areas has also been reported. Guanabenz, was noted to have anti-prion activity through its anti-PFAR activity (D. Tribouillard-Tanvier et al., 2008 PLoS One 3, e1981); its activity in protecting against protein misfolding based on its PP1c/PPP1R15A phosphatase complex inhibitory activity was also reported. Based on its action on protein misfolding, guanabenz has been investigated in a randomized Phase 2 study in ALS patients. Guanabenz has been described as reducing NMDA-induced currents and NMDA-induced cytosolic Ca²⁺ load (Ruiz et al. Int. J. Mol. Sci. 2020, 21, 6088).

The close derivative 2-(2-chlorobenzylidene)hydrazinecarboximidamide, referred to as icerguastat, IFB-088 or sephin1, but devoid of hypotensive activity, also displays a PP1c/PPP1R15A phosphatase complex inhibitory activity which protects against protein misfolding. This compound displays therapeutic potential to treat Charcot Marie Tooth (CMT) disease and ALS. IFB-088/icerguastat was shown to reduce NMDA-induced cytosolic Ca²⁺ load (Ruiz et al. Int. J. Mol. Sci. 2020, 21, 6088). The ability of IFB-088 and some benzylideneguanidine derivatives of formula (I) to inhibit NMDAR have been evaluated by Ring et al. (Bioorganic Medicinal Chem. 2013 (21) 1764-1774) by assessing their ability to displace radiolabeled MK-801; the compounds displayed a high IC₅₀ of about 5 microM to displace this NMDAR channel blocker.

Further benzylideneguanidine compounds and their therapeutic applications associated with protein misfolding stress are known from EP2943467, WO2016/001389, WO2016/001390 or WO2017/021216. EP109465 (CNRS) displays guanabenz derivatives as PFAR ligands to treat prion-diseases. WO2002/011715 (Melacure) displays benzylideneguanidine compounds as melanocortin receptor ligands for disease treatments. WO2005/031000 (Acadia Pharmaceuticals) displays benzylideneguanidine compounds as neuropeptide FF receptor 2 agonists for the treatment of neuropathic pain.

To exploit NMDA receptor antagonists as possible treatments, it is necessary to develop new NR2B selective negative allosteric modulators with a reduced side-effect liability and able to target the central and peripheral nervous system. Prior art publications do not describe benzylideneguanidine derivatives of formula (I) having NR2B subunit selective NMDA receptor antagonist effect which are devoid of psychotic and negative symptoms, as well as cognitive impairment, that resemble those present in schizophrenia. This is the objective of the invention.

In addition, to exploit NMDA receptor antagonists as possible treatments, it is necessary to develop new NR2B selective negative allosteric modulators having a good oral bioavailability, with the ability to cross the blood brain barrier and to target the central and peripheral nervous system. Prior art NR2B subunit NMDAR antagonist such as Ifenprodil had such limitations, which do not appear in benzylidene aminoguanidine derivatives of formula (I) of the invention. This is the objective of the invention.

### SUMMARY OF THE INVENTION

NR2B subunit selective NMDA antagonism can be achieved with compounds that specifically bind to, and act on, an allosteric modulatory site of the NR2B subunit containing receptors. This binding site can be characterized by displacement (binding) studies with specific radioligands, such as [¹²⁵1]-ifenprodil [J.Neurochem., 61, 120-126 (1993)] or [³H]-Ro 25,6981 [J. Neurochem., 70, 2147-2155 (1998)].

Surprisingly it was found that the benzylideneguanidine derivatives of formula (I) of the present invention are functional antagonists of NMDA receptors, which target the NMDA receptors primarily via binding to or near to the ifenprodil binding site on NR2B subunit, and not via binding to the pore channel, like MK801 as suggested by Ring et al. 2013 (Ring et al. Bioorganic Medicinal Chem. 2013 (21) 1764-1774).

These benzylideneguanidine derivatives of formula (I) of the present invention protect cells against glutamate-induced excitotoxicity via inhibiting calcium influx by antagonizing NR2B subunit containing NMDAR. Therefore, they are believed to be selective NR2B subunit antagonists or NR2B negative allosteric modulators. Thus, the benzylideneguanidine derivatives of formula (I) of the present invention can inhibit the downstream neurotoxic cascades and are able to reduce the formation of reactive oxygen species (ROS) for example.

The benzylideneguanidine derivatives of formula (I) of the present invention do not have the limitations of NMDAR antagonists of the prior art, and are devoid of psychotic and negative symptoms, as well as cognitive impairment, that resemble those present in schizophrenia.

### DETAILED DESCRIPTION OF THE INVENTION

Abbreviations

| | |
|---|---|
| AD | Alzheimer's Disease |
| ALS | Amyotrophic Lateral Sclerosis |
| CMAP | Compound Muscle Action Potential |
| CMT | Charcot-Marie-Tooth Disease |
| CNS | Central Nervous System |
| HD | Huntington's Disease |
| MS | Multiple Sclerosis |
| NMDA | N-methyl-D-aspartate |
| NMDAR | N-methyl-D-aspartate receptor |
| PNS | Peripheral Nervous System |
| PBA | Pseudobulbar affect |
| PD | Parkinson's Disease |
| ROS | Reactive Oxygen Species |

### Definitions

Unless clearly indicated otherwise, the following terms as used herein have the meanings indicated below.

As used herein, the term “alkyl” includes both saturated straight chain and branched alkyl groups which may be substituted (mono- or poly-) or unsubstituted. Preferably, the alkyl group is a C₁₋₂₀ alkyl group, more preferably a C₁₋₁₅, more preferably still a C₁₋₁₂ alkyl group, more preferably still, a C₁₋₆ alkyl group, more preferably a C₁₋₃ alkyl group. Particularly preferred alkyl groups include, for example, methyl, ethyl, propyl, isopropyl, butyl, isobutyl, tert-butyl, pentyl and hexyl. Preferably, the alkyl group is unsubstituted. The term “alkoxy,” unless otherwise specified, refers to a moiety of the structure —O-alkyl, wherein alkyl is as defined above.

As used herein, the term “aryl” refers to a C₆₋₁₂ aromatic group which may be substituted (mono- or poly-) or unsubstituted. Typical examples include phenyl and naphthyl etc. Suitable substituents include, for example, one or more R¹⁰ groups. The term “aryloxy,” unless otherwise specified, refers to a moiety of the structure —O-aryl, wherein aryl is as defined above.

The term “excitotoxicity” refers to a pathological process of neuronal damage and destruction or neurotoxicity, by hyperactivation by glutamic acid or glutamate and its analogues (all being excitatory neurotransmitters). These neurotransmitters activate neuronal excitatory receptors such as NMDA and AMPA receptors. These excitotoxins such as NMDA (N-methyl-D-aspartate) and kainic acid, or glutamate in excessive concentration, by binding to these receptors cause a massive entry of calcium ions into the cell. Ca²⁺ in turn activates a number of enzymes including phospholipases C, endonucleases and proteases such as calpain. These enzymes then degrade cellular structures: cytoskeleton, cell membrane, DNA, leading to neurotoxicity. Increased intracellular Ca²⁺ concentration also leads to an increased formation of ROS which will provoke damages to multiple cellular organelles and processes, which can ultimately disrupt normal physiology. This physiopathological mechanism is incriminated in several neurological diseases such as spinal cord trauma, brain trauma, strokes, acquired deafness (by ototoxicity linked to overexposure to noise), neurodegenerative diseases of the central nervous system, such as MS, AD, ALS, PD, HD, epilepsy and fibromyalgia.

The term “halo” or “halogen,” refers to chloro, bromo, iodo, or fluoro. The term “haloalkyl” refers to an alkyl radical having one or more hydrogen atoms replaced by a halogen atom. The term “hydroxyl” refers to the functional group (—OH).

The term “treatment or prevention,” as used herein in the context of treating or preventing a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviation of symptoms of the condition, amelioration of the condition, and cure of the condition. Treatment as a prophylactic measure (i.e., prevention) is also included, for example, use with patients who have not yet developed the condition, but who are at risk of developing the condition.

Herein, the term “treating” includes abrogating, substantially inhibiting, slowing or reversing the progression of a disease, substantially ameliorating clinical symptoms of a disease or disorder or substantially improving the survival of a patient. The term «preventing» refers to preventing or delaying the onset of a neurodegenerative disorder, and/or of the appearance of the symptoms thereof. For example, treatment or prevention of neuropathic pain includes: preventing the onset of neuropathic pain, inhibiting the progress of neuropathic pain, reducing the rate of progress of neuropathic pain, reducing the incidence of neuropathic pain, reducing the severity of neuropathic pain, alleviating one or more symptoms of neuropathic pain, amelioration of neuropathic pain, cure of neuropathic pain, etc.

The term “therapeutically-effective amount,” or “effective amount” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

The term “Patient” or “Subject” refers to an animal, such as a mammal, including but not limited to, a human. Hence, the methods and use disclosed herein can be useful in human therapy and veterinary applications. In one embodiment, the patient is a mammal. In another embodiment, the patient is a human.

“Such as” has the same meaning as “such as but not limited to”. Similarly, “include” has the same meaning as “include but not limited to”, while “including” has the same meaning as “including but not limited to”.

The term “psychotic symptom” or “negative symptoms” have the same meaning. Psychotic symptoms include hallucinations, delusions (i.e. false beliefs that don't go away even after they've been shown to be false), disordered forms of thinking. It may also include disorganized or incoherent speech, strange and possibly dangerous behavior, slowed or unusual movements, loss of interest in activities, loss of interest in personal hygiene, problems at school or work and with relationships, cold, detached manner with the inability to express emotion, mood swings or other mood symptoms, such as depression or mania. Psychosis is an abnormal condition of the mind that displays psychotic symptoms; psychosis has several different causes which include mental illness, such as schizophrenia or schizoaffective disorder, bipolar disorder, sensory deprivation and in rare cases, major depression (psychotic depression). Other causes include trauma, sleep deprivation, some medical conditions, certain medications, and drugs such as cannabis, hallucinogens, and stimulants.

The term “cognitive impairment” as used herein, pertains to a description of someone's condition, which have trouble with things like memory or paying attention; they might have also trouble speaking or understanding and difficulties in recognizing people, places or things.

The term “overactivation of NMDA receptor” is used herein to refer to abnormally high levels of signaling activity of NMDAR on CNS and/or PNS cells (e.g. neurons, astrocytes, oligodendrocytes, Schwann cells).

### DESCRIPTION OF THE EMBODIMENTS

The present invention relates to certain benzylideneguanidine compounds which are negative allosteric modulator of NMDAR NR2B subunit and pharmaceutical compositions comprising those compounds, useful for the treatment or prevention of therapeutic indications, as described herein.

According to a first object, the present invention is directed to a method of selectively inhibiting the subunit 2B (NR2B) of the N-methyl-D-aspartate (NMDA) receptor in a cell having NMDA receptor subunit 2B (NR2B)-containing NMDA receptors, the method comprising treating the cell with an effective amount of a compound of general formula (I):

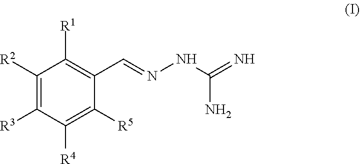

and the (Z) and/or (E) isomers thereof, or a tautomer thereof, or pharmaceutically acceptable salts thereof,
wherein: R1, R2, R3, R4, R5 are independently hydrogen, deuterium, halogen, haloalkyl, alkyl, alkoxy, hydroxyl, aryl, or aryloxy,
so as to effect a neuroprotective reduction in the effect of excitotoxic NMDA receptor activity.

According to a preferred embodiment, the cell is selected in the group consisting of a neuron, motor neuron, sensory neuron, Schwann cell, oligodendrocyte, astrocyte.

According to an embodiment, the reduction in the effect of excitotoxic NMDA receptor activity in the cell may be advantageously provided by a reduction of intracellular Ca²⁺ concentration.

According to an alternative or cumulative embodiment, the reduction in the effect of excitotoxic NMDA receptor activity in the cell may be provided by a reduction of reactive oxygen species (ROS) concentration.

According to a further object, it is provided a method of selectively inhibiting the subunit 2B (NR2B) of the N-methyl-D-aspartate (NMDA) receptor in a subject having NMDA receptor subunit 2B (NR2B)-containing NMDA receptors, the method comprising administering an effective amount of a compound of general formula (I):

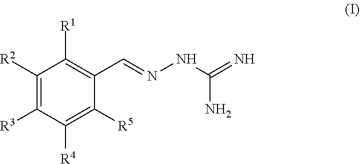

and the (Z) and/or (E) isomers thereof, or a tautomer thereof, or pharmaceutically acceptable salts thereof,
wherein: R1, R2, R3, R4, R5 are independently hydrogen, deuterium, halogen, haloalkyl, alkyl, alkoxy, hydroxyl, aryl, or aryloxy,
in said subject in need thereof.

According to a still further object, it is also provided a method for preventing or treating a disease, disorder, or medical condition caused by overactivation of the N-methyl-D-aspartate (NMDA) receptor containing a subunit 2B (NR2B) by selectively targeting the NR2B subunit of said NMDA receptor, wherein said method comprises administering to a subject in need thereof an effective amount of a compound of general formula (I):

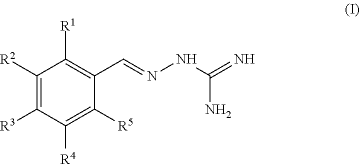

and the (Z) and/or (E) isomers thereof, or a tautomer thereof, or pharmaceutically acceptable salts thereof, wherein: R1, R2, R3, R4, R5 are independently hydrogen, deuterium, halogen, haloalkyl, alkyl, alkoxy, hydroxyl, aryl, or aryloxy.

According to an embodiment, the disease, disorder, or medical condition may be selected from the group consisting of:
(a) depression or a depressive disorder, a major depressive disorder, a treatment-resistant major depressive disorder, post-partum depression, bipolar depression;
(b) anxiety disorder, obsessive compulsive disorder, generalized anxiety disorder, agoraphobia with panic disorder, panic disorder, post-traumatic-stress disorder, social anxiety disorder;
(c) autism or autism spectrum disorder, Asperger's syndrome, or pervasive developmental disorder not otherwise specified (PDD-NOS);
(d) epilepsy, seizure disorder;
(e) migraine, chronic tension type headache (CTTH), migraine with allodynia, chronic headache;
(f) abnormal brain function, selected among Fragile X syndrome, tuberous sclerosis, Down's syndrome and other forms of mental retardation;
(g) withdrawal syndromes, e.g. alcohol, opioids or cocaine;
(h) pain, hyperalgesia, nociception, acute pain, chronic pain, or cancer-related pain;
(i) pain associated with excitotoxicity, preferably with glutamate excitotoxicity, and/or is associated with malfunctioning of glutamatergic neurotransmission;
(j) neuropathic pain;
(k) pseudobulbar affect (PBA).
(l) dyskinesia;
(m) amyotrophic lateral sclerosis (ALS) or bulbar-onset ALS;
(n) Charcot-Marie-Tooth disease;
(o) multiple sclerosis (MS);
(p) Parkinson's disease, atypical parkinsonian disorders (e.g. progressive supranuclear palsy);
(q) Alzheimer disease (AD), dementia, frontotemporal dementias (FTDs), progressive supranuclear palsy (PSP), corticobasal degeneration (CBD).
(r) Huntington's disease (HD);
(s) focal brain injuries from trauma, tumors or strokes;
(t) brain or spinal cord injury, peripheral nervous system injury, cerebral ischemia, head or neuronal trauma, neuronal hemorrhage, neuronal ischemia, reperfusion injury, neuronal injury;
(u) neuronal exposure to a toxic substance, methamphetamine-induced neurotoxicity; (v) stroke, cardiogenic shock, coronary artery bypass graft (CABG) surgery associated neurological damage;
(w) Idiopathic pulmonary fibrosis (IPF) and chronic cough and symptoms thereof.

According to an embodiment, the neuropathic pain is selected from the group consisting of: the peripheral neuropathic pain; central neuropathic pain; chronic neuropathic pain; refractory neuropathic pain; neuropathic pain associated with a metabolic dysfunction, including, for example, diabetes mellitus and pre-diabetes; neuropathic pain associated with diabetes mellitus; neuropathic pain associated with pre-diabetes; neuropathic pain associated with painful polyneuropathy; neuropathic pain associated with painful diabetic neuropathy, including, for example, diabetic peripheral neuropathy; neuropathic pain associated with painful diabetic polyneuropathy; neuropathic pain associated with post-herpetic neuralgia; neuropathic pain associated with trigeminal neuralgia; neuropathic pain associated with occipital neuralgia; neuropathic pain associated with painful radiculopathy, including, for example, lumbar and cervical painful radiculopathy; neuropathic pain associated with an infectious disease, including, for example, herpes zoster (shingles), HIV infection, Lyme disease, diphtheria, and leprosy; neuropathic pain associated with a liver or kidney disorder, including, for example, a chronic liver or kidney disorder, including, for example, liver disease, liver failure, kidney disease, and kidney failure; neuropathic pain associated with an immune or inflammatory disorder, including, for example, Guillain-Barre syndrome, rheumatoid arthritis, lupus, Sjörgren's syndrome, and coeliac disease; neuropathic pain associated with an inherited neuropathy or channelopathy, including, for example, inherited erythromelalgia, paroxysmal extreme pain disorder, and Charcot-Marie-Tooth disease (CMT); neuropathic pain associated with small fiber sensory neuropathy; neuropathic pain associated with a thyroid hormone disorder, including, for example, hypothyroidism; neuropathic pain associated with stroke; neuropathic pain associated with cancer, including, for example, lymphoma and multiple myeloma; neuropathic pain associated with chemotherapy, for example, cancer chemotherapy; neuropathic pain associated with peripheral nerve injury pain; neuropathic pain associated with nerve damage following traumatic injury; neuropathic pain associated with post-traumatic neuropathy; neuropathic pain associated with spinal cord injury, including, for example, spinal cord injury caused by trauma, for example, a road traffic accident; neuropathic pain associated with traumatic peripheral nerve injury; neuropathic pain associated with post-surgery neuropathy (e.g., post-surgery neuropathic pain); neuropathic pain following surgery, including, for example, neuropathic pain following nerve surgery, including, for example, spinal cord surgery; neuropathic pain associated with fibromyalgia; neuropathic pain associated with lower back pain; neuropathic pain associated with carpal tunnel syndrome; neuropathic pain associated with causalgia; neuropathic pain associated with reflex sympathetic dystrophy (RSD); neuropathic pain associated with Complex Regional Pain Syndrome (CRPS), including, for example, Type 1 and Type 2; neuropathic pain associated with amputation; neuropathic pain associated with a neurodegenerative disease, for example, Amyotrophic Lateral Sclerosis and Parkinson's disease; neuropathic pain associated with stroke, including, for example, central post-stroke pain; neuropathic pain associated with syringomyelia; neuropathic pain associated with a demyelinating disease, including, for example, multiple sclerosis, transverse myelitis, and neuromyelitis optica; or idiopathic neuropathic pain.

According to an embodiment, the method of the invention prevents, treats or alleviates pseudobulbar affect (PBA), or symptoms thereof, in a subject selected from the group consisting of patients having Parkinson's disease (PD), or atypical parkinsonian disorders (e.g. progressive supranuclear palsy), amyotrophic lateral sclerosis (ALS), bulbar-onset ALS, multiple sclerosis (MS), Alzheimer disease (AD), dementia, tumors, stroke.

According to an embodiment, the method of the invention prevents, treats or alleviates depression, or symptoms thereof, in a subject selected from the group consisting of patients having Parkinson's disease (PD), or atypical parkinsonian disorders (e.g. progressive supranuclear palsy), Alzheimer disease (AD), Huntington disease (HD), amyotrophic lateral sclerosis (ALS), bulbar-onset ALS, multiple sclerosis (MS), CMT.

According to an embodiment, the method of the invention prevents, treats or alleviates dyskinesia, or the symptoms thereof, in a subject selected from the group consisting of patients having Parkinson's disease (PD), or atypical parkinsonian disorders (e.g. progressive supranuclear palsy).

According to an embodiment, the method of the invention does not simultaneously involve side effects chosen from psychotic effect, cognitive impairment and symptoms associated with schizophrenia.

According to an embodiment, in formula (I) above:
R1, R3 and R5 are independently chosen from H, Cl, F, Br and OH;
R2=R4=H.

According to a preferred embodiment, the compounds may be chosen from the group consisting in:
2-(2,6-Dichlorobenzylidene)hydrazinecarboximidamide
2-(2-Chlorobenzylidene)hydrazinecarboximidamide
2-(2-Chloro-4-fluoro benzylidene)hydrazinecarboximidamide
2-(2-Chloro-6-fluorobenzylidene)hydrazinecarboximidamide
2-(2-Bromobenzylidene)hydrazinecarboximidamide
2-(2-Fluorobenzylidene)hydrazinecarboximidamide
2-(2,4-Difluorobenzylidene)hydrazinecarboximidamide
2-(2,6-Difluorobenzylidene)hydrazinecarboximidamide acetate salt
2-(2,4-Dichlorobenzylidene)hydrazinecarboximidamide acetate salt
2-(2,3-Dichlorobenzylidene)hydrazinecarboximidamide
2-(2,3,4-Trichlorobenzylidene)hydrazinecarboximidamide
2-(3,4,5-Trichlorobenzylidene)hydrazinecarboximidamide
2-(2,4,6-Trifluorobenzylidene)hydrazinecarboximidamide acetate salt
2-(2,4,5-Trifluorobenzylidene)hydrazinecarboximidamide
2-(2,6-Difluoro-4-chlorobenzylidene)hydrazinecarboximidamide
2-(2,4-Dichloro-3-fluorobenzylidene)hydrazinecarboximidamide
2-(2-Chloro-4,6-difluorobenzylidene)hydrazinecarboximidamide
2-(2-Chloro-4,5-difluorobenzylidene)hydrazinecarboximidamide
2-(2-Chloro-4-hydroxybenzylidene) hydrazinecarboximidamide
2-(2-Chloro-3-methylbenzylidene)hydrazinecarboximidamide
2-(2-Chloro-4-methylbenzylidene)hydrazinecarboximidamide
2-(2-Chloro-5-methylbenzylidene)hydrazinecarboximidamide
2-(2,4-Dichloro-6-fluorobenzylidene)hydrazinecarboximidamide
2-(2,6-Dichloro-4-fluorobenzylidene)hydrazinecarboximidamide
2-(2,3-Dichloro-4-fluorobenzylidene)hydrazinecarboximidamide
2-(2-Chloro-3, 5-difluorobenzylidene)hydrazinecarboximidamide
2-(3,4-Dichloro-6-fluorobenzylidene)hydrazinecarboximidamide
2-(3,5-Dichloro-4-fluorobenzylidene)hydrazinecarboximidamide
2-(2,4-Dichloro-5-fluorobenzylidene)hydrazinecarboximidamide
2-(2,3,5-Drichlorobenzylidene)hydrazinecarboximidamide
2-(3,4,5-Drifluorobenzylidene)hydrazinecarboximidamide
2-(2,3,4-Drifluorobenzylidene)hydrazinecarboximidamide
   and the (Z) and/or (E) isomers thereof, or a tautomer thereof, or pharmaceutically acceptable salts thereof.

More preferably, the compound of formula (I) may be chosen from:

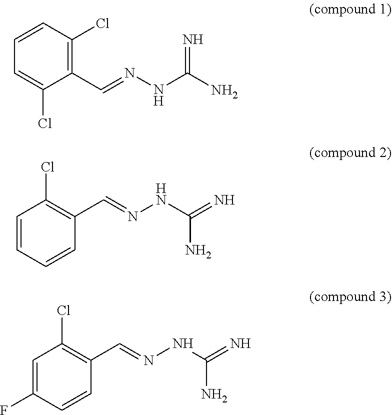

and the (Z) and/or (E) isomers thereof, or a tautomer thereof, or pharmaceutically acceptable salts thereof.

Still more preferably, the compound of formula (I) is chosen from:
compound 2 of formula:

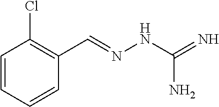 [SMILES: N=C(N)N/N=C/c1ccccc1Cl]

and the (Z) and/or (E) isomers thereof, or a tautomer thereof, or pharmaceutically acceptable salts thereof; and
the (Z) isomer of compound 1 of formula:

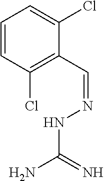 [SMILES: N=C(N)N/N=C\c1c(Cl)cccc1Cl]

In another embodiment, the invention relates to a compound of general formula (I):

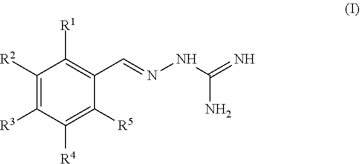

and the (Z) and/or (E) isomers thereof, or a tautomer thereof, or pharmaceutically acceptable salts thereof,
wherein: R1, R2, R3, R4, R5 are independently hydrogen, deuterium, halogen, haloalkyl, alkyl, alkoxy, hydroxyl, aryl, or aryloxy,
for use for the treatment of disease, disorder, or medical condition mediated by NR2B-containing NMDA receptor activity, such the ones described in this patent application.

According to a further object, the present invention concerns the following compounds per se selected from the group consisting in:
2-(2,4,6-Trifluorobenzylidene)hydrazinecarboximidamide acetate salt
2-(2,6-Difluoro-4-chlorobenzylidene)hydrazinecarboximidamide
2-(2-Chloro-4,6-difluorobenzylidene)hydrazinecarboximidamide
2-(2-Chloro-4-hydroxybenzylidene)hydrazinecarboximidamide
2-(2,4-Dichloro-6-fluorobenzylidene)hydrazinecarboximidamide
2-(2,6-Dichloro-4-fluorobenzylidene)hydrazinecarboximidamide
2-(2-Chloro-3,5-difluorobenzylidene)hydrazinecarboximidamide
   and the (Z) and/or (E) isomers thereof, or a tautomer thereof, or pharmaceutically acceptable salts thereof.

### Therapeutic Indications

The Applicant has demonstrated that the compounds of the invention have potential therapeutic applications in treating and/or preventing a disease, disorder, or medical condition mediated by NR2B-containing NMDA receptor activity.

Unless specifically provided, the present invention preferably relates to human patients.

### Neurodegeneration

According to one embodiment, said disease, disorder, or medical condition mediated by NR2B-containing NMDA receptor activity, is caused by NMDA receptor mediated excitotoxicity.

According to a second embodiment, said disease, disorder, or medical condition mediated by NR2B-containing NMDA receptor activity, is a neurological disease whereby glutamate mediated excitotoxicity is involved in neuronal cell death.

In one embodiment, the invention relates to the inhibition of the ionic channels, such as calcium channels, that are activated by exogenous chemical substances or by endogenous chemical substances (e.g. glutamate), that lead to the neurodegeneration.

In another embodiment, the invention relates to the treatment, slowing down, reduction, decrease and/or prevention of neurodegeneration.

The invention relates to the reduction of neuronal cell injury associated with glutamate excitotoxicity and wherein said glutamate excitotoxicity is mediated by overstimulation of NMDA receptors and wherein said glutamate excitotoxicity is associated with disease, disorder, or medical condition mediated by NR2B-containing NMDA receptor activity such the ones described in this patent application.

### Depression

Depression is a psychiatric disorder characterized by a mental state of low mood and aversion to activity that affects millions of people worldwide. People experiencing depression may have feelings of dejection, hopelessness and suicidal thoughts. Available anti-depressant drugs targeting serotonin and noradrenalin neurotransmission, triggers undesired side effects and provide therapeutic benefits only after a long period of administration. Anti-depression-like effects have been demonstrated by several NMDA receptor antagonists in different animal models and in clinic (Ates-Alagoz Z & Adejare A. NMDA receptor antagonists for treatment of depression. Pharmaceuticals. 2013; 6:480-499). NR2B specific NMDAR antagonists were shown to display antidepressant effects (Henter I D et al. Glutamatergic modulators of depression. Harv. Rev. Psychiatry 2018; 26(6); 307-319).

According to a preferred embodiment, the invention relates to a method for preventing or treating a disease, disorder, or medical condition mediated by NR2B-containing NMDA receptor activity, wherein said method comprises administering to a subject in need of such a treatment an effective amount of a compound of general formula (I) and the (Z) and/or (E) isomers thereof, or a tautomer thereof, or pharmaceutically acceptable salts thereof, wherein the disease, disorder, or medical condition is selected from the group consisting of depression or a depressive disorder, a major depressive disorder, a treatment-resistant major depressive disorder, post-partum depression, bipolar depression, and suicidal ideation.

In one embodiment, the depression is major depressive disorder.

In one embodiment, the depression is treatment-resistant major depressive disorder.

In one embodiment, the depression is post-partum depression.

In one embodiment, the depression is bipolar depression.

### Seizure Disorders, Epilepsy and Abnormal Brain Function

Epilepsy is a group of neurological disorders due to abnormal electrical activity in the brain characterized by recurrent epileptic seizures which vary from brief to long periods of vigorous shaking. NMDAR NR2B subunits were shown to contribute to epilepsy-associated pathological and biochemical events (Zhu X. et al. NMDA receptor NR2B subunits contribute to PTZ-kindling-induced hippocampal astrocytosis and oxidative stress. Brain Res. Bulletin 2015).

According to a preferred embodiment, the invention relates to a method for preventing or treating a disease, disorder, or medical condition mediated by NR2B-containing NMDA receptor activity selected from the group consisting of seizure disorder, epilepsy, Lennox-Gastaut syndrome, Sturge-Weber syndrome, tuberous sclerosis and Infantile spasm syndrome (ISS).

In one embodiment, the seizure disorder is epilepsy.

In one embodiment, the seizure disorder is Lennox-Gastaut syndrome.

In one embodiment, the seizure disorder is Sturge-Weber syndrome In one embodiment, the seizure disorder is tuberous sclerosis.

The invention also pertains to a method for preventing or treating abnormal brain function mediated by NR2B-containing NMDA receptor activity, in particular wherein said abnormal brain function is selected among Fragile X syndrome, Down's syndrome and other forms of mental retardation.

### Anxiety Disorder

Anxiety and trauma-related disorders, including post-traumatic-stress disorder, are associated with excessive fear reactions, often including an inability to extinguish learned fear, increased avoidance behavior, as well as altered cognition and mood. NMDAR have been associated in regulating these fear-related behaviors (Radulovic J. et al. N-Methyl D-Aspartate Receptor subunit signaling in fear extinction. Psychoparmacology. 2019; 236(1): 239-250).

According to a preferred embodiment, the invention relates to a method for preventing or treating a disease, disorder, or medical condition mediated by NR2B-containing NMDA receptor activity, selected from the group consisting of anxiety disorder, obsessive compulsive disorder, generalized anxiety disorder, agoraphobia with panic disorder, panic disorder, social anxiety disorder and post-traumatic-stress disorder. In preferred embodiment, the anxiety disorder is post-traumatic-stress disorder (PTSD). In another embodiment, the invention relates to a method for preventing, treating or alleviating symptoms of anxiety in particular wherein said subject is selected from the group consisting of patients having Amyotrophic Lateral Sclerosis, multiple sclerosis, Parkinson's disease, atypical parkinsonian disorders, Huntington's disease, Alzheimer's disease, Charcot Marie Tooth disease.

### Autism Spectrum Disorders

Autism spectrum disorders (ASD) refers to a broad range of conditions characterized by social deficits and repetitive behaviors, speech and nonverbal communication. ASD are associated with abnormal imbalances and abnormalities in neuronal excitatory and inhibitory synapses. NMDAR dysfunction have been associated with ASD (Lee E. J et al. NMDA receptor dysfunction in autism spectrum disorders. Curr Opin Pharmacol. 2015; 20:8-13).

According to a preferred embodiment, the invention relates to a method for preventing or treating a disease, disorder, or medical condition mediated by NR2B-containing NMDA receptor activity, selected from the group consisting of autism spectrum disorder, autism, Asperger's syndrome, Childhood Disintegrative Disorder, pervasive developmental disorder not otherwise specified (PDD-NOS) and Rett syndrome.

In one embodiment, the autism spectrum disorder is autism.

In one embodiment, the autism spectrum disorder is Asperger's syndrome.

In one embodiment, the autism spectrum disorder is Childhood Disintegrative Disorder.

In one embodiment, the autism spectrum disorder is Rett syndrome.

In one embodiment, the autism spectrum disorder is PDD-NOS.

### Migraine

A migraine is a common health condition usually characterized by a moderate or severe headache felt as a throbbing pain on one side of the head. Many people also have symptoms such as feeling sick, being sick and increased sensitivity to light or sound. There are several types of migraine, including (i) migraine with aura—where there are specific warning signs just before the migraine begins, such as seeing flashing lights, (ii) migraine without aura—the most common type, where the migraine happens without the specific warning signs, and (iii) migraine with aura without headache, also known as silent migraine—where an aura or other migraine symptoms are experienced, but a headache does not develop. There's no cure for migraines, but a number of treatments are available to help reduce the symptoms, painkillers (e.g. paracetamol, ibuprofen), triptans and anti-emetics. Recent developments suggest that the specific inhibition of GluN2B-containing NMDARs might be effective in preventing migraine pain (Crivellaro G. et al. Specific activation of GIuN1-N2B NMDA receptors underlies facilitation of cortical spreading depression in a genetic mouse model of migraine with reduced astrocytic glutamate clearance. Neurobiology of Disease 2021 156:105419).

According to a preferred embodiment, the invention relates to a method for preventing or treating a disease, disorder, or medical condition mediated by NR2B-containing NMDA receptor activity, selected from the group consisting of migraine, migraine with aura, migraine without aura, silent migraine, retinal migraine, migraine with allodynia, familial hemiplegic migraine, chronic headache, chronic tension type headache (CTTH).

In one embodiment, the migraine is migraine with aura.

In one embodiment, the migraine is migraine without aura.

In one embodiment, the migraine is silent migraine.

In one embodiment, the migraine is migraine with allodynia.

In one embodiment, the migraine is familial hemiplegic migraine.

In one embodiment, the migraine is retinal migraine.

In one embodiment, the migraine is chronic headache.

### Drug Intoxication and Withdrawal

Drugs of abuse (e.g., alcohol, cocaine, opioids, . . . ) exert some of their effects on the central nervous system by affecting glutamatergic transmissions, particularly via NMDAR (Landa L. Implication of NMDA receptors in behavioural sensitization to psychostimulants: a short review. Eur J Pharmacol. 2014; 5:730:77-81). The NR2B subunit of NMDA receptor has been identified as possible central regulators of many addictive behaviors such as alcohol dependence (Nagy J. The NR2B subunit of NMDA receptor: a potential target for the treatment of alcohol dependence. Curr Drug Targets CNS Neurol Disord. 2004 Jun.; 3(3):169-79), cocaine dependance (Smaga I. et al. Enhancement of the GIuN2B subunit of glutamatergic NMDA receptors in rat brain areas after cocaine abstinence. J Psychopharmacol. 2021; 35(10):1226-1239).

According to a preferred embodiment, the invention relates to a method for preventing or treating a disease, disorder, or medical condition mediated by NR2B-containing NMDA receptor activity, selected from the group consisting of drug intoxication, drug withdrawal syndromes, addictive behaviors associated with drugs of abuse, suppressing dependence to drug of abuse, habituation or addiction on a dependence-producing drug; drugs are selected among alcohol, nicotine, marijuana, opioids, phenyclidene, psychostimulants such as amphetamines (e.g. methamphetamine) and cocaine, barbiturates such as pentobarbitone and benzodiazepines such as temazepam, diazepam and flunitrazepam. Prescription opioids are for example morphine, methadone, oxycodone, hydrocodone-Acetaminophen, pseudoephedrine-Hydrocodone, hydromorphone, fentanyl, codeine, methadone, oxymorphone hydrochloride, meperidine, tramadol, carfentanil, buprenorphine. Illegal opioids are for example heroin.

### Pain

Neuropathic pain is pain caused by damage or disease affecting the somatosensory system; it appears after nerve and spinal cord injuries and in certain diseases, producing a debilitation of the patient and a decrease in the quality of life. Neuropathic pain is affecting 7%-8% of the European population. Neuropathic pain may result from disorders of the peripheral nervous system (PNS) or the central nervous system (CNS). Thus, neuropathic pain may be divided into peripheral neuropathic pain, central neuropathic pain, or mixed (peripheral and central) neuropathic pain. CNS pain is found in spinal cord injury, multiple sclerosis and some strokes for example, while PNS pain is often found in peripheral neuropathies caused by diabetes, metabolic disorders, viral infection (herpes zoster, HIV . . . ), nutritional deficiencies, toxins, remote manifestations of malignancies, immune mediated disorders and physical trauma to a nerve trunk. Neuropathic pain is also common in cancer as a direct result of cancer on peripheral nerves (e.g., compression by a tumor), or as a side effect of chemotherapy (chemotherapy-induced peripheral neuropathy), radiation injury or surgery.

The involvement of NR2B-containing NMDARs in neuropathic pain is established (Aiyer et al. Clin J Pain. A systematic review of NMDA receptor antagonists for treatment of neuropathic pain in clinical practice. 2017; Qu et al. Exp. Neurology. Role of the spinal cord NR2B-containing NMDA receptors in the development of neuropathic pain 2009; 215:298-307).

According to a preferred embodiment, the invention relates to a method for preventing or treating a disease, disorder, or medical condition mediated by NR2B-containing NMDA receptor activity, selected from the group consisting of pain, hyperalgesia, nociception, acute pain, chronic pain, cancer-related pain and neuropathic pain.

According to a preferred embodiment, said disease, disorder, or medical condition is selected from the group consisting of pain, hyperalgesia, nociception, acute pain, chronic pain, cancer-related pain and neuropathic pain.

In one embodiment, the pain is hyperalgesia. According to a preferred embodiment, hyperalgesia is selected in the group comprising opioid-induced hyperalgesia, hyperalgesia induced by other analgesics, preferably analgesics acting on the glutamate neurotransmission, or hyperalgesia induced by a chemotherapeutic agent or any other drug.

According to a preferred embodiment, the invention relates to a method for preventing, treating or alleviating symptoms of pain wherein said subject is selected from the group consisting of patients having neuropathic pain.

In one embodiment, the neuropathic pain is peripheral neuropathic pain.

In one embodiment, the neuropathic pain is central neuropathic pain.

In one embodiment, the neuropathic pain is chronic neuropathic pain.

In one embodiment, the neuropathic pain is refractory neuropathic pain.

In one embodiment, the neuropathic pain is neuropathic pain associated with a metabolic dysfunction, including, for example, diabetes mellitus and pre-diabetes.

In one embodiment, the neuropathic pain is neuropathic pain associated with painful polyneuropathy.

In one embodiment, the neuropathic pain is neuropathic pain associated with painful diabetic neuropathy, including, for example, diabetic peripheral neuropathy.

In one embodiment, the neuropathic pain is neuropathic pain associated with post-herpetic neuralgia, trigeminal neuralgia, or occipital neuralgia.

In one embodiment, the neuropathic pain is associated with painful radiculopathy, including, for example, lumbar and cervical painful radiculopathy.

In one embodiment, the neuropathic pain is neuropathic pain associated with an infectious disease, including, for example, herpes zoster (shingles), HIV infection, Lyme disease, diphtheria, and leprosy.

In one embodiment, the neuropathic pain is neuropathic pain associated with a liver or kidney disorder, including, for example, a chronic liver or kidney disorder, including, for example, liver disease, liver failure, kidney disease, and kidney failure.

In one embodiment, the neuropathic pain is neuropathic pain associated with an immune or inflammatory disorder, including, for example, Guillain-Barre syndrome, rheumatoid arthritis, lupus, Sjogren's syndrome, and coeliac disease.

In one embodiment, the neuropathic pain is neuropathic pain associated with an inherited neuropathy or channelopathy, including, for example, inherited erythromelalgia, paroxysmal extreme pain disorder, and Charcot-Marie-Tooth disease (CMT).

In one embodiment, the neuropathic pain is neuropathic pain associated with small fibre sensory neuropathy.

In one embodiment, the neuropathic pain is neuropathic pain associated with a thyroid hormone disorder, including, for example, hypothyroidism.

In one embodiment, the neuropathic pain is neuropathic pain associated with stroke.

In one embodiment, the neuropathic pain is neuropathic pain associated with cancer, including, for example, lymphoma and multiple myeloma.

In one embodiment, the neuropathic pain is neuropathic pain associated with chemotherapy, for example, cancer chemotherapy or with chemotherapy-induced peripheral neuropathy.

In one embodiment, the neuropathic pain is neuropathic pain associated with peripheral nerve injury pain.

In one embodiment, the neuropathic pain is neuropathic pain associated with nerve damage following traumatic injury.

In one embodiment, the neuropathic pain is neuropathic pain associated with post-traumatic neuropathy.

In one embodiment, the neuropathic pain is neuropathic pain associated with spinal cord injury, including, for example, spinal cord injury caused by trauma, for example, a road traffic accident.

In one embodiment, the neuropathic pain is neuropathic pain associated with traumatic peripheral nerve injury.

In one embodiment, the neuropathic pain is neuropathic pain associated with post-surgery neuropathy (e.g., post-surgery neuropathic pain).

In one embodiment, the neuropathic pain is neuropathic pain following surgery, including, for example, neuropathic pain following nerve surgery, including, for example, spinal cord surgery.

In one embodiment, the neuropathic pain is neuropathic pain associated with fibromyalgia.

In one embodiment, the neuropathic pain is neuropathic pain associated with lower back pain.

In one embodiment, the neuropathic pain is neuropathic pain associated with carpal tunnel syndrome.

In one embodiment, the neuropathic pain is neuropathic pain associated with causalgia.

In one embodiment, the neuropathic pain is neuropathic pain associated with reflex sympathetic dystrophy.

In one embodiment, the neuropathic pain is neuropathic pain associated with Complex Regional Pain Syndrome (CRPS), including, for example, Type 1 and Type 2.

In one embodiment, the neuropathic pain is neuropathic pain associated with amputation.

In one embodiment, the neuropathic pain is neuropathic pain associated with a neurodegenerative disease, for example, Parkinson's disease and ALS.

In one embodiment, the neuropathic pain is neuropathic pain associated with stroke, including, for example, central post-stroke pain.

In one embodiment, the neuropathic pain is neuropathic pain associated with syringomyelia.

In one embodiment, the neuropathic pain is neuropathic pain associated with a demyelinating disease, including, for example, multiple sclerosis, transverse myelitis, and neuromyelitis optica.

In one embodiment, the neuropathic pain is idiopathic neuropathic pain.

According to a preferred embodiment, the invention relates to preventing, treating, or alleviating symptoms of pain in a subject in need of such a treatment having ALS.

According to a preferred embodiment, the invention relates to preventing, treating, or alleviating symptoms of pain in a subject in need of such treatment having CMT.

According to a preferred embodiment, the invention relates to preventing, treating, or alleviating symptoms of hyperalgesia in a subject in need of such treatment having CMT.

According to a preferred embodiment, the invention relates to preventing, treating, or alleviating hyperalgesia-induced neuropathic pain in CMT patients, preferably in CMT1A patients.

According to a preferred embodiment, the invention relates to preventing, treating, or alleviating symptoms of pain in a subject in need of such treatment having MS.

According to a preferred embodiment, the invention relates to preventing, treating, or alleviating symptoms of pain in a subject in need of such treatment having PD.

### Pseudobulbar Affect (PBA)

Pseudobulbar affect (PBA) is a medical condition that affects people living with certain neurologic conditions or brain injury, causing involuntary, sudden, and frequent episodes of crying and/or laughing that are disproportionate to the emotion being experienced. PBA has been also referred to as pathological laughing and crying, affective lability, emotional incontinence, emotionalism, and involuntary emotional expression disorder. PBA occurs in the setting of neurological diseases such as ALS, MS, dementia, PD, atypical parkinsonian disorders (e.g., progressive supranuclear palsy), focal brain injuries from trauma, tumors and strokes (estimates range between 11 to 52%). PBA affects up to 49% of patients with ALS and is more prevalent in patient with the bulbar form of the disease (Gallagher et al. Pathological Laughter and crying in ALS: a search for their origin. Acta Neurol. Scand. 1989; 80(2):114-117). Recent studies suggest a lifetime prevalence of PBA of approximately 10% to 74% in Alzheimer's disease, 10% in MS patients and is associated with more severe intellectual deterioration, physical disability, and neurological disability (Schiffer et al. Review of pseudobulbar affect including a novel and potential therapy. J. Neuropsychiatry Clin Neurosci. 2005; 17(4):447-454). PBA can have significant impact on patient's quality of life.

Dextromethorphan, an uncompetitive NMDAR antagonist, combined with quinidine sulfate (Q) is the only treatment of PBA approved by US FDA and EMA.

According to a preferred embodiment, the invention relates to a method for preventing, treating or alleviating pseudobulbar affect (PBA), or symptoms thereof, in a subject in need of such a treatment, wherein said subject is selected from the group consisting of patients having:
a) amyotrophic lateral sclerosis (ALS) or bulbar-onset ALS; or
b) multiple sclerosis (MS); or
c) atypical parkinsonian disorders (e.g. progressive supranuclear palsy); or
d) focal brain injuries from trauma, tumors or strokes.

### Dementia and Alzheimer Disease (AD)

Dementia is a general term for loss of memory, language, problem-solving and other thinking abilities that are severe enough to interfere with daily life. AD is the most common cause of dementia. Memantine, low-affinity NMDAR channel blocker, has been used in the treatment of moderate to severe AD (Liu J. The Role of NMDA Receptors in Alzheimer's Disease. Front Neurosci. 2019; 13:43). Tau mutation A152T (hTauAT), a risk factor for frontotemporal dementias (FTD)-spectrum disorders including progressive supranuclear palsy (PSP) and corticobasal degeneration (CBD) causes excitotoxicity mediated by NR2B-containing NMDA receptors due to enhanced extracellular glutamate (Decker J M. The Tau/A152T mutation, a risk factor for frontotemporal-spectrum disorders, leads to NR2B receptor-mediated excitotoxicity. EMBO Reports (2016) 17: 552-569) According to a preferred embodiment, the invention relates to a method for preventing or treating a disease, disorder, or medical condition mediated by NR2B-containing NMDA receptor activity, wherein the disease, disorder, or medical condition is selected from the group consisting of Alzheimer's disease, dementia, frontotemporal dementias (FTDs), progressive supranuclear palsy (PSP) and corticobasal degeneration (CBD).

### Amyotrophic Lateral Sclerosis (ALS)

ALS is a fatal neurodegenerative disease with results from selective loss of upper and lower motor neurons. There is compelling evidence that both direct and indirect glutamate toxicity contribute to the pathology of motor neuron degeneration. Memantine, a non-competitive NMDAR antagonist, has been shown to protect neurons against NMDA- or glutamate induced toxicity in vitro and in ALS animal model (Wang et al. Memantine prolongs survival in an amyotrophic lateral sclerosis mouse model. Eur J Neurosc. 2005; 22:2376-2380). According to a preferred embodiment, the invention relates to preventing or treating a disease, disorder, or medical condition mediated by NR2B-containing NMDA receptor activity selected from the group consisting of ALS, bulbar-onset ALS, spinal-onset ALS.

### Multiple Sclerosis (MS)

MS is a neurodegenerative disease caused by autoimmune response against myelin in the central nervous system. Glutamate excitotoxicity is a pathophysiological process believe to play roles in MS pathophysiology, like in myelin degradation, blood-brain-barrier disruption, neurovascular injury, cell death and axonal degeneration. Non-selective NMDAR antagonists, such as memantine and MK-801, and selective antagonist of the NR2B-containing NMDARs, Ro25-6981, were effective in modulation of disease in MS animal models (Farjam et al. Inhibition of NR2B-containing N-Methyl-D-Aspartate Receptors (NMDARs) in experimental autoimmune encephalomyelitis, a model of multiple sclerosislran J Pharm Res. 2014 Spring; 13(2):695-705.). According to a preferred embodiment, the invention relates to preventing or treating a disease, disorder, or medical condition mediated by NR2B-containing NMDA receptor activity selected from the group consisting of multiple sclerosis.

### Dyskinesia and Parkinson's Disease (PD)

Dyskinesia is the term used to describe unintended, involuntary and uncontrollable movements which include twitches, jerking, twisting or simple restlessness. Dyskinesia is a symptom of several medical disorders that are distinguished by their underlying cause. In PD for example, dyskinesia is related to the long-term use of certain medications, including levodopa. Less commonly, dyskinesia can also occur when levodopa is just starting to take effect or when it is wearing off and is known as ‘diphasic dyskinesia’. Non-selective NMDAR antagonists, such as MK-801, has been shown to be effective in modulation of levodopa-induced dyskinesia (Wang X S et al. Modulation of CaMKIIa-GIuN2B interaction in levodopa-induced dyskinesia in 6-OHDA-lesioned Parkinson's rats. Biomed Pharmacother 2018 107:769-776). Huntington's disease (HD) patients are also displaying dyskinesia, named chorea.

According to a preferred embodiment, the invention relates to a method for preventing, treating or alleviating dyskinesia or chorea in a subject selected from the group consisting of patients having Parkinson's disease, atypical parkinsonian disorders and Huntington's disease. Atypical parkinsonian disorders include multiple system atrophy (MSA), progressive supranuclear palsy (PSP), corticobasal degeneration (CBD), frontotemporal lobar degeneration (FTLD), Dementia with Lewy bodies (DLB).

According to a preferred embodiment, the dyskinesia is a levodopa-induced dyskinesia.

According to a preferred embodiment, the chorea is chorea of Huntington or HD. As user herein, dyskinesia and chorea have the same meaning.

### Neuronal Damages, Stroke

In response to PNS injury, Schwann cells de-differentiate and acquire the ability to migrate and proliferate. Activated Schwann cells carry out functions that are essential for nerve repair, including phagocytosis of debris, secretion of trophic factors and deposition of provisional extracellular matrix proteins. NMDAR NR1 and NR2B subunits are expressed in Schwann cells and are upregulated in sciatic nerves following crush injury in rat model.

These results define the NMDAR as a Schwann cell signaling receptor for protein ligands and a major regulator of Schwann cell physiology, which may be particularly important in peripheral nervous system (PNS) injury.

According to a preferred embodiment, the invention relates to a method for preventing or treating a disease, disorder, or medical condition mediated by NR2B-containing NMDA receptor activity, selected from the group consisting of
a) brain or spinal cord injury, PNS injury, cerebral ischemia, head or neuronal trauma, neuronal hemorrhage, neuronal ischemia, reperfusion injury, neuronal injury; or
b) neuronal exposure to a toxic substance, methamphetamine-induced neurotoxicity; or
c) stroke, cardiogenic shock, coronary artery bypass graft (CABG) surgery associated neurological damage.

### Idiopathic Pulmonary Fibrosis (IPF) and Chronic Cough

IPF is a serious chronic disease that affects the tissue surrounding the air sacs, or alveoli, in the lungs. The most common symptoms of IPF are shortness of breath and cough. NR2B-selective NMDAR antagonist Ifenprodil has been shown to be effective in a Phase 2a study in patients with IPF (Algernon Pharmaceuticals, Vancouver, British Columbia). According to a preferred embodiment, the invention relates to preventing or treating a disease, disorder, or medical condition mediated by NR2B-containing NMDA receptor activity selected from Idiopathic pulmonary fibrosis (IPF) and chronic cough.

### Treatment and Prevention

As discussed above, the present invention relates to certain compounds and pharmaceutical compositions comprising those compounds, which are useful for the treatment or prevention of disease, disorder, or medical condition, as described herein. The compounds may be administered, for example, at or shortly after the time of disease, disorder, or medical condition is diagnosed or detected to prevent or mitigate the development of disease, disorder, or medical condition. Alternatively, the compounds may be administered during the course of disease disorder, or medical condition.

### Administration

The compounds of the present invention may be adapted for oral, rectal, nasal, intrabronchial, topical (including buccal, sublingual and ophthalmic administration), vaginal or parenteral (including subcutaneous, intramuscular, intravenous, intraarterial and intradermal), intraperitoneal or intrathecal administration. Preferably the formulation is an orally administered formulation. The formulations may conveniently be presented in unit dosage form, i.e., in the form of discrete portions containing a unit dose, or a multiple or sub-unit of a unit dose. By way of example, the formulations may be in the form of tablets and sustained release capsules and may be prepared by any method well known in the art of pharmacy.

### Dosage

A person of ordinary skill in the art can easily determine an appropriate dose of one of the instant compounds to administer to a subject. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

In accordance with this invention, an effective amount of a compound of the invention may be administered to target a particular condition or disease. Of course, this dosage amount will further be modified according to the type of administration of the compound. For example, to achieve an “effective amount” for acute therapy, parenteral administration of a combination of the invention is preferred. The precise amount thereof which is therapeutically effective, and the route by which such compound is best administered, is readily determined by one of ordinary skill in the art by comparing the blood level of the agent to the concentration required to have a therapeutic effect.

The compounds of this invention may also be administered to the patient, in a manner such that the concentration of drug is sufficient to achieve one or more of the therapeutic indications disclosed herein.

No unacceptable toxicological effects are expected when compounds of the present invention are administered in accordance with the present invention. The compounds of this invention, which may have good bioavailability, may be tested in one of several biological assays to determine the concentration of a compound which is required to have a given pharmacological effect.

### Salts

The compounds of the invention can be present as salts, in particular pharmaceutically and veterinarily acceptable salts.

Pharmaceutically acceptable salts of the compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. hydrohalic acids such as hydrochloride, hydrobromide and hydroiodide, sulfuric acid, phosphoric acid sulphate, bisulphate, hemisulphate, thiocyanate, persulphate and sulphonic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with amino acids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Salts which are not pharmaceutically or veterinary acceptable may still be valuable as intermediates.

Preferred salts include, for example, acetate, trifluoroacetate, lactate, gluconate, citrate, tartrate, maleate, malate, pantothenate, adipate, alginate, aspartate, benzoate, butyrate, digluconate, cyclopentanate, glucoheptanate, glycerophosphate, oxalate, heptanoate, hexanoate, fumarate, nicotinate, palmoate, pectinate, 3-phenylpropionate, picrate, pivalate, proprionate, tartrate, lactobionate, pivolate, camphorate, undecanoate and succinate, organic sulphonic acids such as methanesulphonate, ethanesulphonate, 2-hydroxyethane sulphonate, camphorsulphonate, 2-naphthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids.

### Tautomers

In all aspects of the present invention previously discussed, the invention includes, where appropriate all tautomers of the compounds of the invention. The person skilled in the art will recognise compounds that possess tautomeric characteristics. The corresponding tautomers may be isolated/prepared by methods known in the art.

Compounds of formula (I) may include the tautomer forms of formula:

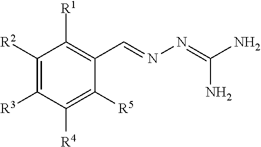

As an illustrative example, tautomer forms of compound 1 (guanabenz) are:

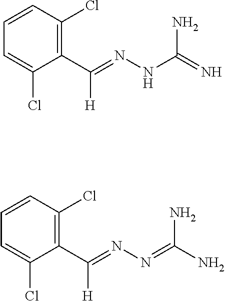 [SMILES: N=C(N)N/N=C/c1c(Cl)cccc1Cl ; NC(N)=N/N=C/c1c(Cl)cccc1Cl]

As a further illustrative example, tautomers of compound 2 are:

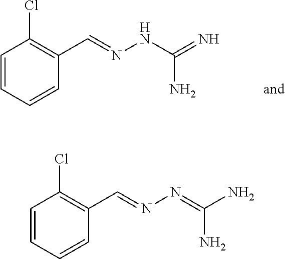 [SMILES: N=C(N)N/N=C/c1ccccc1Cl ; "and" ; NC(N)=N/N=C/c1ccccc1Cl]

### Geometric Isomers

Some of the compounds of the invention may exist as geometric isomers. They may possess one or more geometric centres and so may exist in two or more geometric forms. The double bond between benzylidene and guanidine moieties (—HC═N— bond) enables the compounds of formula (I) to exist as E- or Z-isomer. For some compounds, a high barrier for thermal isomerization exists between the two isomers; thus the spontaneous isomerization of compound 1 (guanabenz) in solid and solution states is practically insignificant (Xie et al. J Pharma Biomed Analysis. LC-MS/MS determination of guanabenz E/Z isomers and its applications to in vitro and in vivo DMPK profiling studies 2021, 205). As an illustrative example, geometric isomer forms of compound 1 are:

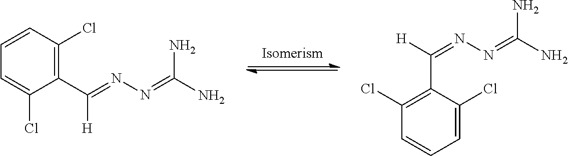 [SMILES: NC(N)=N/N=C/c1c(Cl)cccc1Cl ; NC(N)=N/N=C\c1c(Cl)cccc1Cl]

(Deepika K. et al. Crystal Growth & Design. Geometrical Isomerism in Guanabenz Free Base Synthesis, Characterization, Crystal Structure and Theoretical Studies 2019).

The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

The present invention also includes all suitable isotopic variations of the agent or a pharmaceutically acceptable salt thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, fluorine and chlorine such as ²H, ³H, ¹³C, ¹⁴C ¹⁵N, ¹⁷, ¹⁸, ¹⁸F and ³⁶Cl, respectively. Certain isotopic variations of the agent and pharmaceutically acceptable salts thereof, for example, those in which a radioactive isotope such as ³H or ¹⁴C, or non-radioactive isotope such as ¹³C, is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. For example, the invention includes compounds of general formula (I) where any hydrogen atom has been replaced by a deuterium atom. Isotopic variations of the agent of the present invention and pharmaceutically acceptable salts thereof of this invention can generally be prepared by conventional procedures using appropriate isotopic variations of suitable reagents.

### Formulations

For use according to the present invention, the compounds or physiologically acceptable salts or other physiologically functional derivatives thereof, described herein, may be presented as a pharmaceutical formulation, comprising the compounds or physiologically acceptable salt or other physiologically functional derivative thereof, together with one or more pharmaceutically acceptable carriers therefore and optionally other therapeutic and/or prophylactic ingredients. The carrier(s) must be acceptable in the sense of being compatible with the other ingredients of the formulation and not deleterious to the recipient thereof. The pharmaceutical compositions may be for human or animal usage in human and veterinary medicine.

Examples of such suitable excipients for the various forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilizing agent(s), buffer(s), flavoring agent(s), surface active agent(s), thickener(s), preservative(s) (including anti-oxidants) and the like, and substances included for the purpose of rendering the formulation isotonic with the blood of the intended recipient.

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

Pharmaceutical formulations include those suitable for oral, topical (including dermal, buccal, ocular and sublingual), rectal or parenteral (including subcutaneous, intradermal, intramuscular and intravenous), nasal, intra-ocularly and pulmonary administration e.g., by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration or sprinkled on food. The granules may be packaged, e.g., in a sachet.

Formulations suitable for oral administration wherein the carrier is a liquid may be presented as a solution or a suspension in an aqueous or non-aqueous liquid, or as an oil-in-water liquid emulsion.

Formulations for oral administration in the present invention may be presented as: discrete units such as capsules, gellules, drops, cachets, pills or tablets each containing a predetermined amount of the active agent; as a powder or granules; as a solution, emulsion or a suspension of the active agent in an aqueous liquid or a non-aqueous liquid; or as an oil-in-water liquid emulsion or a water-in-oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropyl methylcellulose, sucrose and starch; fillers and carriers, for example corn starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier.

Formulations for oral administration include controlled release dosage forms, e.g., tablets wherein an active compound is formulated in an appropriate release—controlling matrix, or is coated with a suitable release—controlling film.

Other forms of administration comprise solutions or emulsions which may be injected intravenously, intraarterially, intrathecally, subcutaneously, intradermally, intraperitoneally, intra-ocularly, topical, peri-ocularly or intramuscularly, and which are prepared from sterile or sterilisable solutions. Injectable forms typically contain between 10-1000 mg, preferably between 10-250 mg, of active ingredient per dose.

The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch.

Pharmaceutical formulations suitable for parenteral administration include sterile solutions or suspensions of an active compound in aqueous or oleaginous vehicles.

Injectable preparations may be adapted for bolus injection or continuous infusion.

Alternatively, an active compound may be in powder form which is constituted with a suitable vehicle, such as sterile, pyrogen-free water, before use.

An active compound may also be formulated as long-acting depot preparations, which may be administered by intramuscular injection or by implantation, e.g., subcutaneously or intramuscularly. Depot preparations may include, for example, suitable polymeric or hydrophobic materials, or ion-exchange resins.

Pharmaceutically acceptable carriers are well known to those skilled in the art and include, but are not limited to, 0.1 M and preferably 0.05 M phosphate buffer or 0.8% saline.

Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Preservatives and other additives may also be present, such as, for example, antimicrobials, antioxidants, chelating agents, inert gases and the like.

According to a further aspect of the invention, there is provided a process for the preparation of a pharmaceutical or veterinary composition as described above, the process comprising bringing the active compound(s) into association with the carrier, for example by admixture.

In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of general formula (I) in conjunction or association with a pharmaceutically or veterinarily acceptable carrier or vehicle.

### FIGURES

The present invention is further described with reference to the following figures, wherein:

FIG. 1 shows the reduction of intracellular calcium flux upon treatment with different concentrations of compounds 1 (FIG. 1A), 2 (FIG. 1B) and 3 (FIG. 1. C) in HEK293 cell line expressing recombinant NMDAR stressed with glutamate. Data shown was normalized to the maximal and minimal response observed in the presence of control ligand (MK-801) and vehicle respectively (y-axis) and is plotted against the corresponding compound concentration in nM in log 10 scale (x-axis).

FIG. 2 shows the ability of different concentrations of compounds 1, 2 and 3 to displace radiolabeled ligand Ifenprodil. Biochemical assay results are presented as the percent inhibition of specific binding. Compound 1 (FIG. 2A), compound 2 (FIG. 2B), compound 3 (FIG. 2C). Red Square/line: Ifenprodil, Blue circle/line: Compound.

FIG. 3 shows the recovery of toe spreading following nerve injury upon treatment with compound 3. Results are expressed as a percentage of control condition as mean+/−SEM (n=10-11/group). Plain line: Compound 3 (1.5 mg/kg/day). Dotted line: Vehicle.

FIG. 4 shows the effect of compounds 2 on the regeneration of sciatic nerves after mechanical stress assessed by electromyography and histology analysis. A-B: Electromyography (EMG) profile following sciatic nerve injury. The latency (ms) and the amplitude (mV) of the CMAP were recorded in gastrocnemius muscle after stimulation of the sciatic nerve. Results are expressed as mean+/−SEM (n=10-11/group). Dotted line: Vehicle; Plain line: Compound 2 (3 mg/kg twice daily).

C-F: Histological analysis of peripheral nerves after sciatic nerve injury. Results are expressed as mean+/−SEM (n=4-5/group). White bar: contralateral vehicle treated; black bar: ipsilateral vehicle treated; hatched bar: ipsilateral compound 2 (3 mg/kg twice daily) treated. (C) myelin thickness expressed in percentage over contralateral nerve; (D) percentage of myelinated axons per sciatic nerve compared to contralateral nerve; (E) myelin thickness in micrometer (F) Picture of sciatic nerve section.

FIG. 5 shows the effect of 16 weeks treatment of PMP22 transgenic rats, a model of CMT1A, with compound 2 (3 mg/kg QD) on thermal hyperalgesia assessed with hot plate test (52° C.). 4-week-old WT and CMT1A rats were orally administered with vehicle or compound 2 at 3 mg/kg s.i.d. for 16 weeks (n=8-12 rats per condition). CMT1A rats and WT rats were placed into a glass cylinder on a hot plate adjusted to 52° C. The latency to paw lifting, shaking or licking has been recorded. Data are expressed in seconds (s) as mean+SEM. *p<0.05 vs CMT1A vehicle by Kruskal-Wallis followed by Dunn's post-test. White bar: WT rats vehicle treated, Black bar: PMP22 transgenic rats vehicle treated, hatched bar: PMP22 transgenic rats treated with compound 2 (2.29 mg/kg QD).

FIG. 6 shows the increase of primary motoneurons viability by compound 2 upon glutamate stress. (FIG. 6A) Compound 2 at 100 nM and 500 nM increase the viability of wild type rat primary motoneurons following 5 μM glutamate treatment for 20 minutes. (FIG. 6B) Compound 2 from 10 nM to 5 μM increases the viability of primary motoneurons from SOD1^{G93A} transgenic rats following 5 μM glutamate treatment for 20 minutes. Data are expressed are expressed as a percentage of control, as a mean−/+SEM. White bar: vehicle treated; black bar: glutamate treated only; hatched bar: glutamate stressed and compound 2 (different concentrations) treated.

FIG. 7 shows a reduction of reactive oxygen species (ROS) in primary SOD1^{G93A} transgenic motoneurons stressed with 5 μM glutamate treatment for 20 minutes and treated with compound 2 at 100 and 500 nM. Data are expressed are expressed as a percentage of control, as a mean−/+SEM. White bar: vehicle treated; black bar: glutamate treated only; hatched bar: glutamate stressed and compound 2 (different concentrations) treated.

FIG. 8 shows a reduction of intracellular calcium flux (FIG. 8A) and ROS concentration (FIG. 8B) upon treatment with different concentrations of compound 2 in primary cortical neurons stressed by NMDA.

White bar: vehicle treated; black bar: NMDA treated only; hatched bar: NMDA stressed and compound 2 treated (different concentrations); grey bar: NMDA stressed and ifenprodil 5 μM treated.

### EXAMPLES

The present invention is further described with reference to the following non-limiting examples.

### Example 1: Chemical Synthesis of Compound 19

Compounds may be prepared by application and adaptation of the procedures disclosed in EP2943467, WO2016/001389, WO2016/001390 or WO2017/021216.

For example, 2-(2-chloro-4-hydroxybenzylidene)hydrazinecarboximidamide was prepared with the following route:

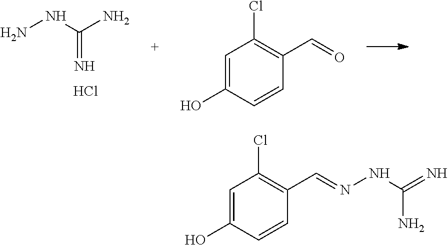

To a solution of 2-chloro-4-Hydroxy-benzaldehyde (2.0 g, 1 eq.) in ethanol (30 ml) was sequentially added Amino guanidine hydrochloride (1 eq.) and sodium acetate (1 eq.) at 25° C. The resulting reaction mixture was heated at 80° C. for next ˜6 hours. Reaction completion was monitored on TLC using dichloromethane/methanol (9/1) as mobile phase. After completion of reaction, the reaction mixture was allowed to cool down to 25° C. and dumped in the saturated solution of NaHC0₃ (100 ml). The resulting precipitate were filtered off under vacuum and washed with water (30 ml). The resulting solid material was triturated with diethyl ether (2×25 ml) and dried under vacuum to provide 2.1 g of 2-(2-chloro-4-hydroxy benzylidene) hydrazinecarboximidamide.

More particularly, the following compounds have been synthesized:

| Compound | | | Profile NMR/LCMS |
|---|---|---|---|
| Number | Structure | Chemical Name | (E isomer) |
| Compound 1 | 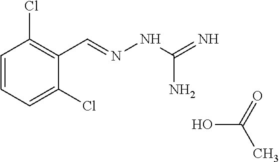 | 2-(2,6- dichlorobenzylidene)hydrazine- carboximidamide acetate salt | guanabenz (commercially available from Merck ref: G110 |
| Compound 2 | 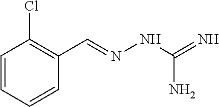 [SMILES: N=C(N)N/N=C/c1ccccc1Cl] | 2-(2- chlorobenzylidene)hydrazine- carboximidamide | icerguastat/sephin1/IFB-088 (as free base) ¹H-NMR (DMSO-d₆): δ (ppm) 5.66 (s, 2H); 6.05 (s broad, 2H); 7.27 (m, 2H); 7.40 (m, 1H); 8.14 (dd, 1H); 8.27 (s, 1H); MS (ESI+): m/z = 197.2 |
| | | | [M + H]⁺. |
| Compound 2A | 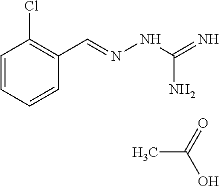 | 2-(2- chlorobenzylidene)hydrazine- carboximidamide acetate salt | icerguastat/sephin1/IFB-088 (as acetate salt) ¹H-NMR (DMSO-d₆): δ (ppm) 1.83 (t, 3H); 6.90 (s broad, 5H); 7.32 (m, 2H); 7.43 (m, 1H); 8.19 (m, 1H); 8.35 (s, 1H); MS (ESI+): m/z = 197.2 [M + H]⁺. |
| Compound 3 | 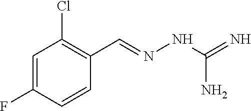 [SMILES: N=C(N)N/N=C/c1ccc(F)cc1Cl] | 2-(2-chloro-4-fluoro- benzylidene)hydrazine- carboximidamide | ¹H-NMR (DMSO-d6): δ (ppm) 5.67 (s, 2H); 6.08 (s broad, 2H); 7.20 (m, 1H); 7.40 (m, 1H); 8.19 to 8.23 (m, 2H); MS (ESI+): m/z = 215.2 [M + H]⁺. |
| Compound 4 | 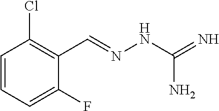 [SMILES: N=C(N)N/N=C/c1c(F)cccc1Cl] | 2-(2-chloro-6- fluorobenzylidene)hydrazine- carboximidamide | ¹H-NMR (DMSO-d₆): δ (ppm) 5.84 (brs, 2H); 5.88 (brs, 2H); 7.18-7.35 (m, 3H); 8.16 (s, 1H); MS (ESI+): m/z = 215.4 [M + H]⁺. |
| Compound 5 | 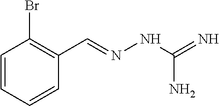 [SMILES: N=C(N)N/N=C/c1ccccc1Br] | 2-(2- bromobenzylidene)hydrazine- carboximidamide | 1H-NMR (DMSO-d6): δ (ppm) 5.07 (s broad, 2H), 5.81 (s broad, 2H), 7.13 (t, 1H), 7.53 (t, 1H), 7.50 (d, 1H), 8.05 (d, 1H), 8.51 (s, 1H); MS (ESI+): m/z = 242.0 [M + H]+. |
| Compound 6 | 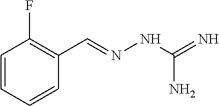 [SMILES: N=C(N)N/N=C/c1ccccc1F] | 2-(2- fluorobenzylidene)hydrazine- carboximidamide | 1H-NMR (DMSO-d6): δ (ppm) 5.69 (s broad, 2H), 6.04 (s broad, 2H), 7.29 (m, 2H), 7.55 (m, 1H), 8.11 (t, 1H), 8.26 (s, 1H); MS (ESI+): m/z = 181.2 [M + H]+. |
| Compound 7 | 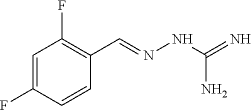 [SMILES: N=C(N)N/N=C/c1ccc(F)cc1F] | 2-(2,4- difluorobenzylidene)hydrazine- carboximidamide | ¹H-NMR (DMSO-d6): δ (ppm) 5.71 (s broad, 2H), 6.09 (s broad, 2H), 7.06 (t, 1H), 7.21 (t, 1H), 8.07 (s, 1H), 8.12 (q, 1H); MS (ESI+): m/z = 199.2 [M + H]⁺. |
| Compound 8 | 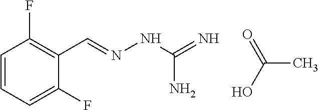 | 2-(2,6- difluorobenzylidene)hydrazine- carboximidamide acetate salt | ¹H-NMR (DMSO-d6): δ (ppm) 1.86 (s, 3h), 6.38 (s broad, 4H), 7.11 (t, 2H), 7.34 (m, 1H), 8.08 (s, 1H); MS (ESI+): m/z = 199.0 [M + H]⁺. |
| Compound 9 | 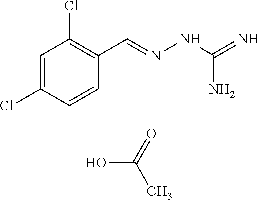 | 2-(2,4- dichlorobenzylidene)hydrazine- carboximidamide acetate salt | ¹H-NMR (MeOD): δ (ppm) 1.95 (s, 3H), 7.40 (dd, 1H); 7.55 (d, 1H); 8.17 (d, 1H); 8.49 (s, 1H), MS (ESI+): m/z = 231.0 [M + H]⁺. |
| Compound 10 | 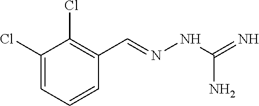 [SMILES: N=C(N)N/N=C/c1cccc(Cl)c1Cl] | 2-(2,3- dichlorobenzylidene)hydrazine- carboximidamide | Commercially available (Raphin) |
| Compound 11 | 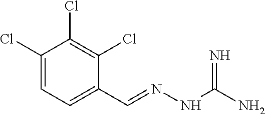 [SMILES: N=C(N)N/N=C/c1ccc(Cl)c(Cl)c1Cl] | 2-(2,3,4- trichlorobenzylidene)hydrazine- carboximidamide | ¹H NMR (400 MHz, DMSO-d₆) δ 8.21 (m, J = 18.0 Hz, 2H), 7.55 (d, J = 8.8 Hz, 1H), 6.30 (s,2H), 5.99 (s,2H); MS (ESI+): m/z = 265.3 [M + H]+. LCMS: 100%, HPLC: 99.36%. |
| Compound 12 | 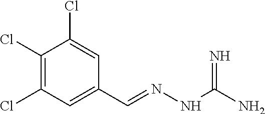 [SMILES: N=C(N)N/N=C/c1cc(Cl)c(Cl)c(Cl)c1] | 2-(3,4,5- trichlorobenzylidene)hydrazine- carboximidamide | ¹H NMR (400 MHz, DMSO-d₆) δ 7.97 (s, 2H), 7.91 (s, 1H), 6.24 (s,2H), 5.75 (s,2H); MS (ESI+): m/z = 265.3 [M + H]+. LCMS: 100%, HPLC: 99.19%. |
| Compound 13 | 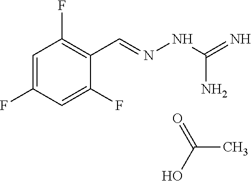 | 2-(2,4,6- trifluorobenzylidene)hydrazine- carboximidamide acetate salt | ¹H-NMR (DMSO-d6): δ (ppm) 1.87 (s, 3h), 6.32 (s broad, 4H), 7.21 (t, 1H), 8.07 (s, 1H); MS (ESI+): m/z = 217.0 [M + H]⁺. |
| Compound 14 | 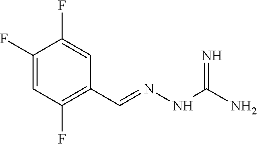 [SMILES: N=C(N)N/N=C/c1cc(F)c(F)cc1F] | 2-(2,4,5- trifluorobenzylidene)hydrazine- carboximidamide | ¹H NMR (400 MHz, DMSO- d₆) δ 8.20 (m, J = 8.0 Hz, 1H), 7.99 (s, 1H), 7.51 (m, J = 6.8 Hz, 1H), 6.20 (s,2H), 5.70 (s,2H); MS (ESI+): m/z = 217.3 [M + H]+. LCMS: 100%, HPLC: 99.87% |
| Compound 15 | 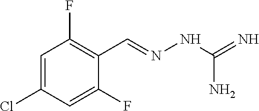 [SMILES: N=C(N)N/N=C/c1c(F)cc(Cl)cc1F] | 2-(2,6-difluoro-4- chlorobenzylidene)hydrazine- carboximidamide | ¹H-NMR (DMSO-d6): δ (ppm) 5.86 (s, 4h), 7.36 (d, 2H), 7.99 (s, 1H); MS (ESI+): m/z = 233.1 [M + H]⁺. |
| Compound 16 | 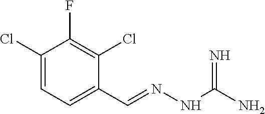 [SMILES: N=C(N)N/N=C/c1ccc(Cl)c(F)c1Cl] | 2-(2,4-dichloro-3- fluorobenzylidene)hydrazine- carboximidamide | ¹H NMR (400 MHz, DMSO- d₆) δ 8.19 (s, 1H), 8.05 (m, J = 10.4 Hz, 1H), 7.49 (m, J = 16.0 Hz, 1H), 6.22 (s,2H), 5.89(s, 2H); MS (ESI+): m/z = 249.3 [M + H]+. LCMS: 99.88%, HPLC: 99.91% |
| Compound 17 | 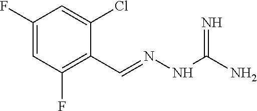 [SMILES: N=C(N)N/N=C/c1c(F)cc(F)cc1Cl] | 2-(2-chloro-,4,6- difluorobenzylidene)hydrazine- carboximidamide | 1H NMR (400 MHz, DMSO- d6) δ 8.28 (m, J = 21.6 Hz, 1H), 8.10 (s, 1H), 7.35 (m, 2H), 5.83 (m, 4H); MS (ESI+): m/z = 233.1 [M + H]+. |
| Compound 18 | 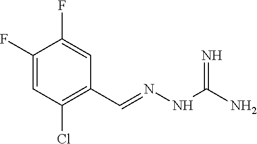 [SMILES: N=C(N)N/N=C/c1cc(F)c(F)cc1Cl] | 2-(2-chloro-,4,5- difluorobenzylidene)hydrazine- carboximidamide | ¹H NMR (400 MHz, DMSO- d₆) δ 8.28 (m, J = 21.6 Hz, 1H), 8.11 (d, J = 2.0 Hz, 1H), 7.67 (m, J = 17.6 Hz, 1H), 6.24 (s,2H), 5.74 (s, 2H); MS (ESI+): m/z = 233 [M + H]+. LCMS: 100%, HPLC: 99.80% |
| Compound 19 | 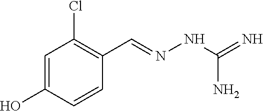 [SMILES: N=C(N)N/N=C/c1ccc(O)cc1Cl] | 2-(2-chloro-4- hydroxybenzylidene)hydrazine- carboximidamide | ¹H-NMR (DMSO-d6): δ (ppm) 5.478 (s, 2H); 5.912 (s broad, 2H); 6.706 (m, 1H); 6.765 (d, 1H); 7.957 (m, 1H); 8.186 (s, 1H), MS (ESI+): m/z = 213.2 [M + H]+. LCMS: 99.82%, HPLC: 99.27% |
| Compound 20 | 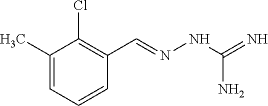 | 2-(2-chloro-3- methylbenzylidene)hydrazine- carboximidamide | ¹H-NMR (DMSO-d₆): δ (ppm) 2.17 (s, 3H); 5.64 (s broad, 2H); 6.03 (s broad, 2H); 7.18 (t, 2H); 7.24 (d, 1H); 7.99 (s, 1H); 8.37 (s, 1H); MS (ESI+): m/z = 210.9 [M + H]⁺. |
| Compound 21 | 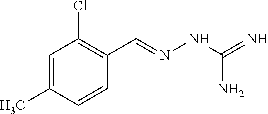 | 2-(2-chloro-4- methylbenzylidene)hydrazine- carboximidamide | ¹H-NMR (DMSO-d₆): δ (ppm) 2.29 (s, 3H); 5.60 (s broad, 2H); 6.00 (s broad, 2H); 7.10 (d, 2H); 7.27 (s, 1H); 8.02 (d, 1H); 8.24 (s, 1H); MS (ESI+): m/z = 210.9 [M + H]⁺. |
| Compound 22 | 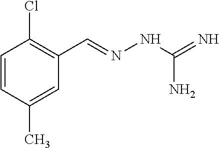 | 2-(2-chloro-5- methylbenzylidene)hydrazine- carboximidamide | ¹H-NMR (DMSO-d₆): δ (ppm) 2.30 (s, 3H); 5.64 (s broad, 2H); 6.06 (s broad, 2H); 7.07 (d, 2H); 7.27 (d, 1H); 7.97 (s, 1H); 8.24 (s, 1H); MS (ESI+): m/z = 210.9 [M + H]⁺. |
| Compound 23 | 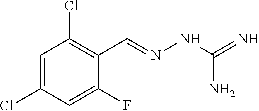 [SMILES: N=C(N)N/N=C/c1c(F)cc(Cl)cc1Cl] | 2-(2,4-dichloro-6- fluorobenzylidene)hydrazine- carboximidamide | LCMS: Method H, 3.13 min, MS: ES+ 249. 1H NMR (400 MHz, DMSO-d6) δ ppm: 5.87 (s, 4H), 7.49-7.52 (m, 2H), 8.11 (s, 1H) |
| Compound 24 | 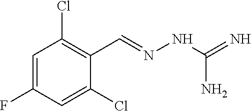 [SMILES: N=C(N)N/N=C/c1c(Cl)cc(F)cc1Cl] | 2-(2,6-dichloro-4- fluorobenzylidene)hydrazine- carboximidamide | LCMS: Method H, 3.115 min, MS: ES+ 249. 1H NMR (400 MHz, DMSO-d6) δ ppm: 5.79- 5.84 (m, 4H), : 7.54-7.56 (d, J = 8.4 Hz, 2H), 8.13 (s, 1H) |
| Compound 25 | 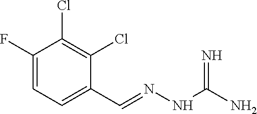 [SMILES: N=C(N)N/N=C/c1ccc(F)c(Cl)c1Cl] | 2-(2,3-dichloro-4- fluorobenzylidene)hydrazine- carboximidamide | 1H NMR (400 MHz, DMSO- d6) δ 8.22 (m, J = 15.2 Hz, 2H), 7.41 (d, J = 17.6 Hz, 1H), 6.18 (s, 2H), 5.77 (s,2H); MS (ESI+): m/z = 249.3 [M + H]+. LCMS: 100%, HPLC: 97.21% |
| Compound 26 | 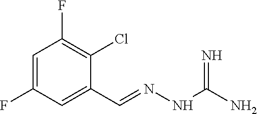 [SMILES: N=C(N)N/N=C/c1cc(F)cc(F)c1Cl] | 2-(2-chloro-3,5- difluorobenzylidene)hydrazine- carboximidamide | ). 1H NMR (400 MHz, DMSO- d6) δ 8.20 (d, J = 2.0 Hz, 1H), 7.95 (d, J = 9.6 Hz, 1H), 7.35 (m, J = 22.0 Hz, 1H), 6.31 (s,2H), 5.86 (s,2H); MS (ESI+): m/z = 233.5 [M + H]+. LCMS: 99.55%, HPLC: 99.83% |
| Compound 27 | 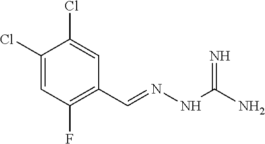 [SMILES: N=C(N)N/N=C/c1cc(Cl)c(Cl)cc1F] | 2-(3,4-dichloro-6- fluorobenzylidene)hydrazine- carboximidamide | 1H NMR (400 MHz, DMSO- d6) δ 8.39 (d, J = 7.2 Hz, 1H), 7.97 (s, 1H), 7.65 (d, J = 10.4 Hz, 1H), 6.25 (s,2H), 5.77 (s, 2H); MS (ESI+): m/z = 249.5 [M + H]+. LCMS: 99.55%, HPLC: 99.38% |
| Compound 28 | 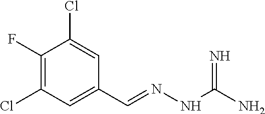 [SMILES: N=C(N)N/N=C/c1cc(Cl)c(F)c(Cl)c1] | 2-(3,5-dichloro-4- fluorobenzylidene)hydrazine- carboximidamide | 1H NMR (400 MHz, DMSO- d6) δ 7.92 (m, J = 14.8 Hz, 2H), 6.16 (s, 2H), 5.62 (s, 2H), MS (ESI+): m/z = 249.3 [M + H]+. LCMS: 100%, HPLC: 100% |
| Compound 29 | 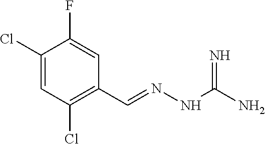 [SMILES: N=C(N)N/N=C/c1cc(F)c(Cl)cc1Cl] | 2-(2,4-dichloro-5- fluorobenzylidene)hydrazine- carboximidamide | ¹H NMR (400 MHz, DMSO- d6) δ 8.25 (d, J = 11.2 Hz, 1H), 8.13 (s, 1H), 7.74 (d, J = 6.8 Hz, 1H), 6.30 (s,2H), 5.81 (s, 2H); MS (ESI+): m/z = 249.3 [M + H]+. LCMS: 100%, HPLC: 100% |
| Compound 30 | 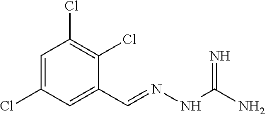 [SMILES: N=C(N)N/N=C/c1cc(Cl)cc(Cl)c1Cl] | 2-(2,3,5- trichlorobenzylidene)hydrazine- carboximidamide | 1H NMR (400 MHz, DMSO- d6) δ 8.25 (d, J = 2.4 Hz, 1H), 8.16 (s, 1H), 7.63 (d, J = 2.4 Hz, 1H), 6.31 (s,2H), 5.85 (s, 2H); MS (ESI+): m/z = 265.2 [M + H]+. LCMS: 100%, HPLC: 100% |
| Compound 31 | 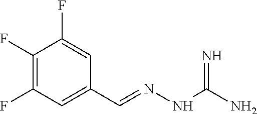 [SMILES: N=C(N)N/N=C/c1cc(F)c(F)c(F)c1] | 2-(3,4,5- trifluorobenzylidene)hydrazine- carboximidamide | 1H NMR (400 MHz, DMSO- d6) δ 7.89 (s, 1H), 7.66 (m, J = 16.4 Hz, 2H), 6.16 (s, 2H), 5.65 (s,2H); MS (ESI+): m/z = 217.3 [M + H]+. LCMS: 100%, HPLC: 99.04% |
| Compound 32 | 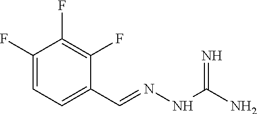 [SMILES: N=C(N)N/N=C/c1ccc(F)c(F)c1F] | 2-(2,3,4- trifluorobenzylidene)hydrazine- carboximidamide | 1H NMR (400 MHz, DMSO- d6) δ 8.20 (s, 1H), 7.93 (m, J = 20.4 Hz, 1H), 7.27 (m, J = 26.0 Hz, 1H), 6.13 (s, 2H), 5.78 (s,2H); MS (ESI+): m/z = 217.3 [M + H]+. LCMS: 100%, HPLC: 99.90% |

### Example 2: Evaluation of Central Nervous (CNS) System Activity Following a Single Oral Administration of Compound 2 as Acetate Salt (Compound 2A) in Rats

The objective of this study was to evaluate the potential effects of compound 2 on CNS activity after a single oral administration to conscious rats. The FOB (Functional Observation Battery) allows the detection of CNS dysfunctions by means of clinical observation and the determination of reactivity to different stimuli, to predict safety profile of the test compounds against CNS. This test is an adaptation of a method described by Mattson J. L. et al (1996, J. Am. Coll. Toxicol., 15, 239).

### Method

A total of 32 male rats (Sprague-Dawley) were allocated to four groups (n=8 animals per group) and received the vehicle (sterile saline solution) or test item (compound 2 as acetate salt, 2A), by a single oral administration at the dose-levels of 1, 3 or 9 mg/kg (of corresponding compound 2 as free base). Animals were not fasted or deprived of water prior to treatment. Assignment to the treatment groups was performed according to a computerized stratified randomization based on the body weight of the animals. The functional observations were performed for all animals once before and approximately 1, 3 and 6 hours after administration. The following parameters have been assessed and graded: touch escape, piloerection, fur appearance, salivation, lacrimation, pupil size (presence of myosis or mydriasis) exophthalmia, reactivity to handling, grooming, palpebral closure, tremors, twitches, convulsions, arousal (hypo- and hyper-activity), ataxia, hypotonia, gait, posture, stereotypy, behavior, breathing, defecation, urination. The following measurements, reflexes and responses have been recorded: touch response, visual stimulus, pupil reflex, auditory startle reflex, tail pinch response, righting reflex, landing foot splay, forelimb grip strength. The study was conducted by CiToxLAB France (BP 563-27005 Evreux, France).

### Results

No clinical signs that could be related to treatment with compound 2 were observed during the assessment of functional observations. No neurologic, autonomic or behavioral changes that could be related to treatment with compound 2 at 1, 3 or 9 mg/kg were observed during the FOB assessment. Under the experimental conditions of this study, single oral administration of compound 2 had no effect on CNS activity up to 9 mg/kg.

### Example 3: Safety Profile of Benzylidene Aminoguanidine Derivatives of Formula (I) in Human

The clinical safety of three benzylidene aminoguanidine derivatives of formula (I) have been assessed in human healthy volunteers; they are devoid of psychotic and negative symptoms, as well as cognitive impairment.

Compound 1 (2-(2,6-dichlorobenzylidene)hydrazinecarboximidamide/quanabenz)

In controlled and open therapeutic trials, none of the adverse events seen with compound 1 (i.e. guanabenz) resembled those present in schizophrenia. Reported compound 1 side effects were drowsiness, dry mouth, dizziness and weakness; cardiovascular side effects were rare, apart from a decrease in heart rate (Holmes et al. Guanabenz, A review of its pharmacodynamic properties and therapeutic efficacy in hypertension. Drugs (1983) 26:212-229).

Compound 2 [2-(2-chlorobenzylidene)hydrazinecarboximidamide/IFB-088/icerquastatel and compound 19 [2-(2-chloro-4-hydroxybenzylidene)hydrazinecarboximidamide].

Compounds 2 and 19, the major human metabolite of compound 2, have similar exposure level in human after repeated administration of compound 2.

The tolerability and pharmacokinetic profile of compound 2 and 19 have been assessed in a randomized, double blind, placebo-controlled study of single ascending doses and multiple ascending doses (NCT03610334 with results). Compound 2 was administered in the form of the acetate salt (compound 2A). All tested doses were well tolerated, without serious adverse events. None of the adverse events seen with compound 2 and 19 resembled those present in schizophrenia. No clinically significant abnormality was reported, neither in vital signs (heart rate, blood pressure) nor in laboratory parameters (blood, liver or renal functions). No clinically significant compound 2 and 19-related hypotension, and dizziness was reported.

### Example 4: NMDAR (NR1A/NR2B) Human Glutamate Ion Channel Cell Based Antagonist Ca²⁺ Flux Assay

### Materials and Methods

The NMDAR (1A/2B) Human Glutamate Ion Channel Cell Based Antagonist Ca²⁺ Flux Assay has been conducted at DiscoverX (DiscoverX Corporation) (assay Nº ITEM 87-1002-1544AN). Briefly, Hek293 cells stably transfected to express NMDAR subunit 1A/2B were seeded into 384-well microplates and incubated at 37° C. Cells were loaded with dye prior to testing. Benzylidene aminoguanidine derivatives of formula (I) was added to cells in the presence of NMDAR antagonist (MK-801) at EC₈₀ concentration. Cells were further incubated for 30-60 minutes at 37° C., and compound activity on calcium flux was measured on a FLIPR Tetra (MDS).

### Results

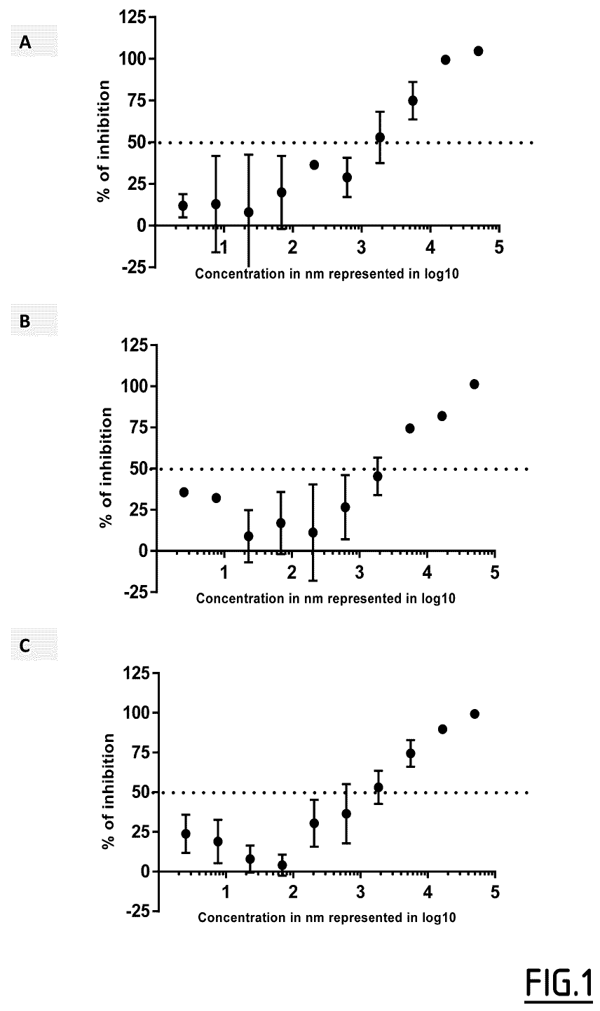

Control compound MK-801 blocked Ca²⁺ flux and displayed an antagonist activity of NMDAR(NR1A/NR2B) with an IC₅₀=69 nM in the assay. Compounds 1, 2 and 3 inhibit Ca²⁺ flux inside cells by antagonizing NMDAR subunit NR1A/NR2B (FIG. 1) and are displaying an IC₅₀ of 625 nM (A), 1156 nM (B) and 702 nM (C) in the assay respectively.

### Example 5: NMDAR Radiolabeled Ligands Displacement Assay

### Materials and Methods

To decipher, the mechanism of action by which the compounds 1, 2 and 3 are displaying their NMDAR antagonist activity, we assess their ability to displace NMDAR radiolabeled ligands. The following radiolabeled ligands were used:
MDL-105,519, a potent and selective antagonist of glycine binding to the NMDAR NR1 subunit;
MK-801, an uncompetitive antagonist that binds inside the ion channel of the NMDAR;
CGP-39653, a potent and selective antagonist of glutamate binding to the NMDAR NR2 subunit;
Ifenprodil, a NR2B selective negative allosteric modulator, that binds in the vicinity of polyamine site.

Assays were conducted at Eurofins (Assay Ref. 232910/233010/234000/SafetyScreen44 Panel) according to previously published methods (Siegel B W et al. (1996) Eur J Pharmacol. 312(3):357-365; Javitt D C and Zukin S R (1989) Interaction of [3H]MK-801 with multiple states of the N-methyl-D-aspartate receptor complex of rat brain. Proc Natl Acad Sci USA. 86(2): 740-744; Reynolds I J, et al (1987) 3H-labeled MK-801 binding to the excitatory amino acid receptor complex from rat brain is enhanced by glycine. Proc Natl Acad Sci USA. 84(21): 7744-7748; Sills M A et al. [“H]CGP39653: a new N-methyl-D-aspartate antagonist radioligand with nanomolar affinity in rat brain. Eur. J. Pharmacol. 1991; 192:19-24; Schoemaker H A & Lang S Z. Binding of [³H]-ifenprodil, a novel NMDA antagonist to a polyamine-sensitive site in the rat cerebral cortex. Eur J Pharmacol. 1990; 176(2):249-250).

### Results

Under the tested conditions, the compounds 1, 2 and 3 were unable to displace radiolabeled ligands MDL-105,519, CGP-39653 and MK-801. Therefore, said compounds does not provide their NMDA antagonist activity by binding to the glycine and glutamate sites and the pore channel, respectively.

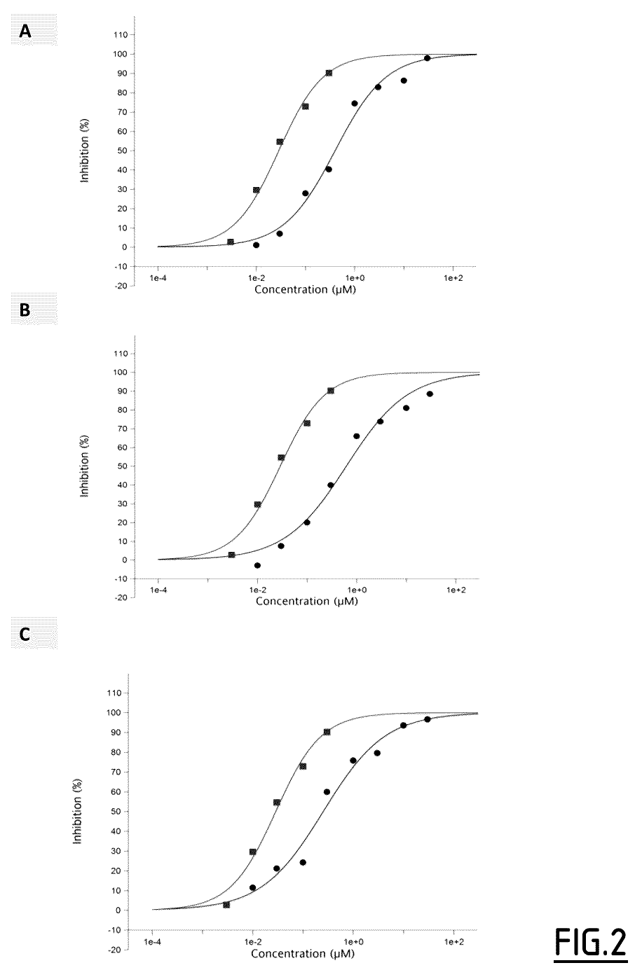

The compounds 1, 2 and 3 were able to displace radiolabeled ligand Ifenprodil and are displaying an IC₅₀ of 400 nM (FIG. 2A), 620 nM (FIG. 2B) and 250 nM (FIG. 2C) in the assay respectively. Thus, the compounds display a NMDA antagonist activity mediated by the binding to, or near to, the Ifenprodil binding site on the NR2B subunit.

### Example 6: Effects of Benzylidene Aminoguanidine Derivatives of Formula (I) on Regeneration of Peripheral Motor Axons, in a Mouse Model of Sciatic Nerve Injury

Sciatic nerve injury on rodents is used to model peripheral nerve regeneration. Sciatic nerve injury, also known as axonotmesis, consist in axonal disruption due to mechanical injury without interruption of connective tissues and basal lamina tubes of Schwann cells (SC). Following injury, the distal part of the axons enters a programmed degenerative process called Wallerian degeneration. Wallerian degeneration is characterized by axonal fragmentation, associated with infiltration of macrophage cells for debris clearance and phenotypic switch of SC. SC play a key role in peripheral nerve regeneration as they coordinate debris removal with macrophages, attract and guide axonal spouts, and finally form new myelin sheaths to ensure correct transmission of electrical signal from neurons.

### Materials and Method

### Sciatic Nerve Injury

6-week-old Swiss (CD-1) male mice were provided by Janvier Labs and housed in an animal facility in a day/night inversed cycle. Sciatic nerve injury was performed as previously described (Henriques et al, 2017 Scientifc report; Bouscary et al, 2019 Frontiers in Pharmacology). Mice were anesthetized with ketamine chlorohydrate (100 mg/kg) and xylazine (10 mg/kg) and placed on a heating pad. Skin was incised and the sciatic nerve exposed at mid-thigh level and lesioned with fine forceps to ensure peripheral injury. The nerve was crushed twice with a haemostatic forceps (width 1.5 mm; Koenig, Strasbourg, France) with a 90-degree rotation between each crush. The skin incision was sutured, and mice were allowed to recover isolated until the end of anesthesia. The hind limb, contralateral to the lesion, served as uninjured control. Analgesia was induced prior surgery and the following days with bunepronorphine (0.1 mg/kg). This surgery resulted in a nerve degeneration over a two-week period followed by localised inflammation of the nerve that lasted for up to four weeks. The loss of nerve function recovered progressively over a 4-5 weeks period after mechanical insult.

### Treatment

Vehicle: Saline (0.9% NaCl, in water)
Doses: Compound 2: 3 mg/kg twice a day;
   Compound 3: 1.5 mg/kg once a daily
Route of administration: Per os (gavage)
Frequency of administration: Twice daily for compound 2 and once daily for compound 3, starting on the day of injury.

### Electromyography

At day 0, on day 7, and on day 14 and day 21 post-surgery, the functionality of the nerve fibers was determined with an electromyography apparatus (Dantec, Natus, France), on the ipsilateral side and contralateral side. Mice were anesthetized with ketamine chlorohydrate (100 mg/kg) and xylazine (10 mg/kg) intraperitoneally. Stimulating needle electrodes were inserted in the sciatic nerve notch and recording needle electrodes were inserted in the gastrocnemius muscle. Reference and ground electrodes were inserted at lower back of the animal and at the base of the paw. The compound muscle action potential (CMAP) was measured: more precisely the amplitude (mV) and the latency (ms) of the action potential were recorded in gastrocnemius muscle after stimulation of the sciatic nerve. The sciatic nerve was stimulated with a single pulse of 0.2 ms at a supramaximal intensity of 12.8 mA. The reference values were less than or equal to 1 ms for the latency and between 40 mA and 60 mA for the amplitude.

### Tissue Collection and Histology

On day 21, mice were deeply anesthetized with ketamine chlorohydrate (100 mg/kg) and xylazine (10 mg/kg) and perfused with cold PBS (3 minutes). Contralateral and ipsilateral tibialis anterior and sciatic nerves collected. Sciatic nerve (n=5 per group, frozen and kept for future analysis) and fixed overnight with 4% glutaraldehyde and maintained in PBS azide 0.02% at +4° C. until use. The nerves were fixed in 1% osmium tetroxide in phosphate buffer for 1h and dehydrated in serial alcohol solutions and embedded in Epon. Embedded tissues were placed at +60° C. during 3 days of polymerization. Transverse sections (1.5 μm of thickness) were generated with a microtome and stained of toluidine blue/fuschine for 30 seconds and dehydrated and mounted in Eukitt. Images were acquired with a confocal laser-scanning microscopy. Morphometric analysis was automatically performed (one section per animal, four different fields) with MetaXpress (Molecular device). The following endpoint parameters were determined (i) number of myelinated axons, (ii) myelin thickness and G factor (axon/fiber diameter ratio).

### Results

### Effect of Compound 3 on Functional Recovery

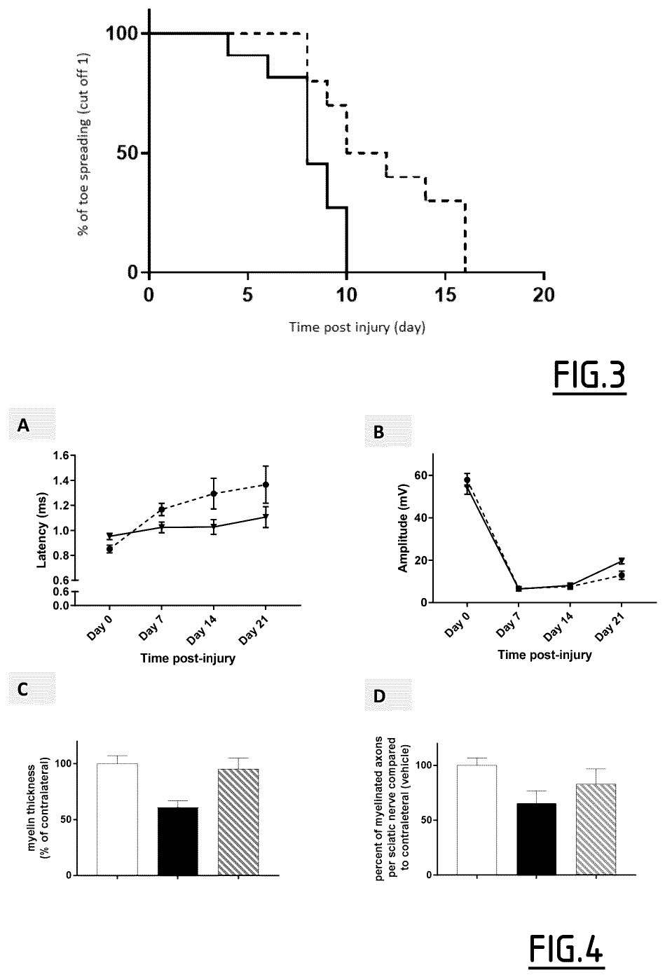

Following nerve injury, spontaneous toe spreading on ipsilateral paw is lost due to denervation of hindlimb muscles. Recovery usually occurs after 10 days post-injury, as shown with vehicle group in FIG. 3. Recovery was faster with Compound 3 (median survival 8 days) when compared to control group, suggesting an improved recovery.

Effect of Compound 2 on Electromyography Profile after Nerve Injury

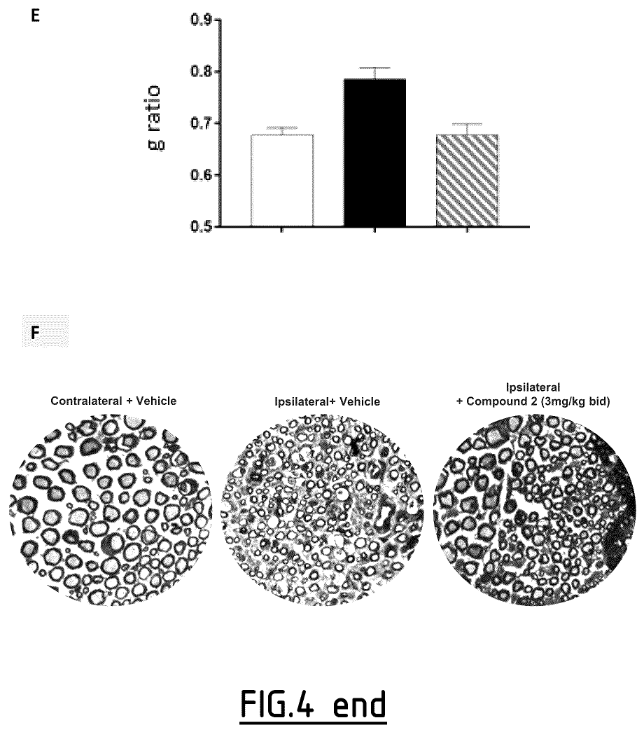

Muscle denervation and demyelination induced profound impairment of compound muscle action potential (CMAP) detected by EMG. CMAP latency and amplitude were analyzed. Latency is defined as the time, in millisecond, between the stimulation, to the onset of the action potential (negative phase of CMAP) (FIG. 4A). Amplitude, in mV, depends on the number of motor axons responding to the stimulation (FIG. 4B). At day 21 post-injury, latency was significantly lower in mice treated with compound 2 (3 mg/kg, twice daily) compared to the control/crushed group. At this time point, compound 2 (3 mg/kg, twice daily) also increased the amplitude of the signal, suggesting an improved regeneration. These results suggest that compound 2 (3 mg/kg, twice daily) reduced axonal degeneration following nerve injury and improved regeneration.

### Effect of Compound 2 on Axons Myelination

Sciatic nerve injury induced a loss of myelinated axons (FIG. 4D) and a reduction of myelin sheath thickness (FIG. 4C-E-F). G-ratio is an index informative for the myelin thickness considering the diameter of axons. Compound 2 did not significantly increase the number of myelinated axons, however, at 3 mg/kg twice a day, it had a strong effect on the myelin thickness of axons that were myelinated (FIG. 4C-D). Evaluation of the G-ratio confirmed the positive effect of compound 2 (3 mg/kg twice a day) (FIG. 4 E). Histology indicates that compound 2 (3 mg/kg, twice daily) improved myelination status of peripheral axons following injury (FIG. 4C-D-E-F).

These results suggest that compound 2 (3 mg/kg, twice daily) reduced axonal degeneration by preventing the myelin degeneration following nerve injury and improved regeneration. Altogether, these results indicate that compound 2 supported axonal regeneration and remyelination in an animal model of peripheral motor injury.

### Example 7: Hot Plate Test

### Materials and Method

Transgenic rat overexpressing PMP22 gene is an established model for Charcot-Marie-Tooth disease subtype 1A (CMT1A). Behavioral and neuromuscular dysfunctions have been evidenced in this model (Sereda et al. Neuron. 1996; 16:1049-1060). The treatment of CMT1A transgenic rat with compound 2 started 4 weeks after birth and lasts for 16 weeks (3 months). Treatment was administrated orally once a day. The hot plate assay was performed after 16-week treatment. The animals were placed into a glass cylinder on a hot plate adjusted to 52° C. (hot) temperature. The latency to paw lifting, shaking or licking was recorded. The cut-off time was set to 45s.

### Results

At 52° C., hyperalgesia was recorded (first sign/reaction) as accredited by the fact that non-treated transgenic CMT1A rats display an average latency of 10.78 seconds which is faster than the latency of 16.02 seconds measured in wild type rats; this latter latency is in accordance to the published data. The response of CMT1A transgenic rats to painful stimuli (i.e. nociceptive response to heat) is faster than normal and corresponds to hyperalgesia (i.e. an over-reaction to painful stimulus).

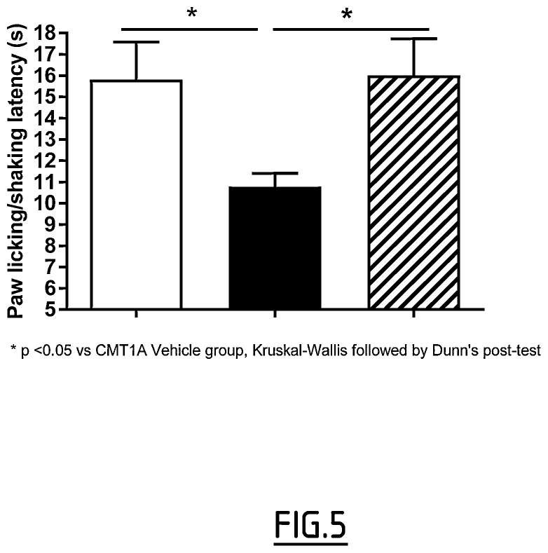

3 months of oral treatment with compound 2 acetate salt (corresponding to 2.29 mg/kg/day of compound 2 as free base) restored the normal latency to pain response in CMT1A transgenic rats, allowing to correct the hyperalgesia symptom (FIG. 5). This result is promising for the treatment of hyperalgesia, and hyperalgesia-induced neuropathic pain, for example in CMT patients.

### Example 8: Effects of Benzylidene Aminoguanidine Derivatives of Formula (I) on the Survival of Wild-Type and SOD1^{G93A} Primary Rat Motoneurons Stressed with Glutamate

### Materials and Method

Spinal cord motor neurons (MNs) from wild-type rat (WT) and SOD1^{G93A} transgenic rat were cultured as described by Boussicault et al., 2020 and Wang et al. 2013. Briefly, pregnant female rats of 14 days gestation were killed using a deep anesthesia with CO₂ chamber and a cervical dislocation. Then, fetuses (E14) were removed from the uterus and immediately placed in ice-cold L15 Leibovitz medium with a 2% penicillin (10,000 U/mL) and streptomycin (10 mg/mL) solution (PS) and 1% bovine serum albumin (BSA). Spinal cords were removed and placed in ice-cold medium of Leibovitz (L15). Spinal cords were treated for 20 min at 37° C. with a trypsin-EDTA solution at a final concentration of 0.05% trypsin and 0.02% EDTA. The dissociation was stopped by addition of Dulbecco's modified Eagle's medium (DMEM) with 4.5 g/liter of glucose, containing DNAse I grade II (final concentration 0.5 mg/ml) and 10% fetal calf serum (FCS). Cells were mechanically dissociated by three forced passages through the tip of a 10-mL pipette. Cells were then centrifuged at 515×g for 10 min at 4° C. The supernatant was discarded, and the pellet was resuspended in a defined culture medium consisting of Neurobasal medium with a 2% solution of B27 supplement, 2 mM of L-glutamine, 2% of PS solution, and 10 ng/mL of brain-derived neurotrophic factor (BDNF). Viable cells were counted in a Neubauer cytometer, using the trypan blue exclusion test. The cells were seeded at a density of 20,000 per well in 96-well plates precoated with poly-L-lysine and were cultured at 37° C. in an air (95%)-CO2 (5%) incubator. The medium was changed every 2 days. The motor neurons were injured with glutamate after 13 days of culture.

On day 13 of culture, compound 2 was applied 1 hour before glutamate application. Glutamate was added to a final concentration of 5 μM diluted in control medium still in presence of the compounds for 20 min. After 20 min, glutamate was washed out and fresh culture medium with compound 2 added for an additional 24 hours.

24 hours after the glutamate application, the supernatants were discarded, and the cells were fixed by a cold solution of ethanol (95%) and acetic acid (5%) for 5 min at −20° C. for immunostaining. Cells were washed twice in PBS, and then are permeabilized and non-specific sites will be blocked with a solution of PBS containing 0.1% of saponin and 1% FCS for 15 min at room temperature. Then, cells were incubated for 2 hours with a mouse monoclonal antibody anti microtubule-associated-protein 2 (MAP-2) at dilution of 1/400 in PBS containing 1% fetal calf serum and 0.1% of saponin. This antibody was revealed with Alexa Fluor 488 goat anti-mouse IgG at the dilution 1/400 in PBS containing 1% FCS, 0.1% saponin, for 1 hour at room temperature.

For each condition, 30 pictures (representative of the whole well area) per well were automatically taken using ImageXpress (Molecular Devices) with 20× magnification. All images were generated by ImageXpress® using the same acquisition parameters. From images, analyses were directly and automatically performed by MetaXpress® (Molecular Devices). The neuron survival was measured by counting neurons MAP-2 stained neurons.

### Results

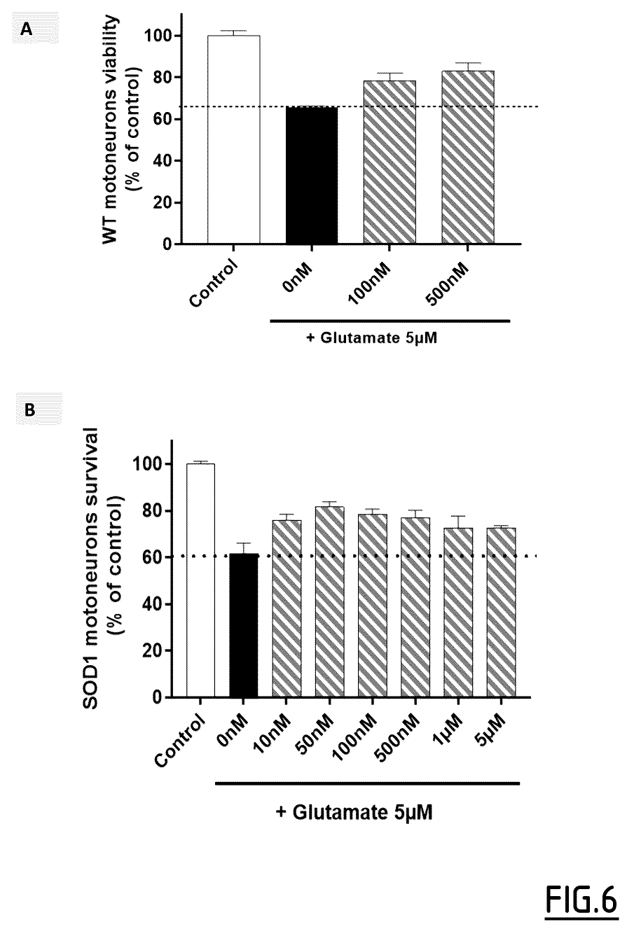

In this in vitro glutamate excitotoxicity assay, nM concentrations of compound 2 improved the survival of primary rat motoneurons from WT (FIG. 6A) or SOD1^{G93A} (FIG. 6B) transgenic rats stressed by 5 μM glutamate. In WT rat motoneurons, compound 2 at 100 nM and 500 nM improved the survival of the motoneurons stressed for 20 minutes by 5 μM of glutamate (FIG. 6A). In SOD1^{G93A} rat motoneurons, compound 2 from 10 nM to 5 μM improved the survival of the primary motoneurons stressed by 5 μM of glutamate (FIG. 6B).

### Example 9: Effects of Benzylidene Aminoguanidine Derivatives of Formula (I) on the ROS Production by Primary Rat Motoneurons Stressed with Glutamate

### Materials and Method

Rat spinal cord motor neurons (MNs) were cultured as described in Example 8. 4 hours after the glutamate application, the cell culture supernatants were discarded. Live cells were incubated with MitoSOX™ Red (marker of ROS generated by the mitochondria) for 10 min at 37° C. The MitoSOX™ reagent is cell-penetrant and will become fluorescent once oxidized by superoxide. Then, cells were incubated for 2 hours with a mouse monoclonal antibody anti microtubule-associated-protein 2 (MAP-2) at dilution of 1/400 in PBS containing 1% fetal calf serum and 0.1% of saponin. This antibody was revealed with Alexa Fluor 488 goat anti-mouse IgG at the dilution 1/400 in PBS containing 1% FCS, 0.1% saponin, for 1 hour at room temperature. Nuclei were counterstained with the fluorescent dye Hoechst (sigma).

For each condition, 30 pictures (representative of the whole well area) per well were automatically taken using ImageXpress (Molecular Devices) with 20× magnification. All images were generated by ImageXpress® using the same acquisition parameters. From images, analyses were directly and automatically performed by MetaXpress® (Molecular Devices). The ROS in MAP-2 positive neurons was measured (overlapping between MAP-2 and mitochondrial ROS in μm²).

### Results

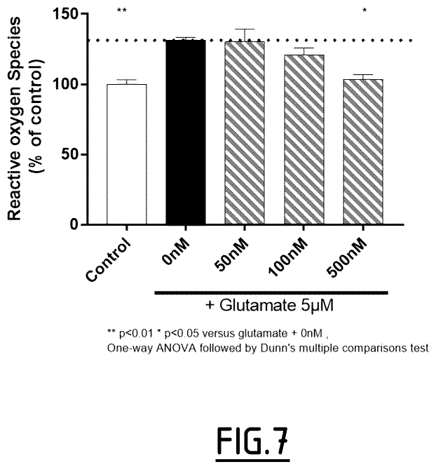

In SOD1^{G93A} transgenic rat motoneurons, compound 2 at 100 nM and 500 nM decreased the amount of ROS produced by motoneurons stressed for 20 minutes by 5 μM of glutamate (FIG. 7).

### Example 10: Effects of Benzylidene Aminoguanidine Derivatives of Formula (I) on the Calcium Flux and the ROS Production in Wild-Type Primary Cortical Neurons Stressed with NMDA

### Materials and Method

Rat cortical neurons were cultured as described by Callizot et al., 2013. Briefly, pregnant female rat (Wistar) of 15 days of gestation were killed using a deep anesthesia with CO₂ chamber and a cervical dislocation. Then, fetuses were collected and immediately placed in ice-cold L15 Leibovitz medium with a 2% penicillin (10,000 U/mL) and streptomycin (10 mg/mL) solution (PS) and 1% bovine serum albumin (BSA). Cortex were treated for 20 min at 37° C. with a trypsin-EDTA solution at a final concentration of 0.05% trypsin and 0.02% EDTA. The dissociation was stopped by addition of Dulbecco's modified Eagle's medium (DMEM) with 4.5 g/L of glucose, containing DNAse I grade II (final concentration 0.5 mg/mL) and 10% fetal calf serum (FCS). Cells were mechanically dissociated by three forced passages through the tip of a 10-mL pipette. Cells were then centrifuged at 515×g for 10 min at 4° C. The supernatant will be discarded, and the pellet will be resuspended in a defined culture medium consisting of Neurobasal medium with a 2% solution of B27 supplement, 2 mmol/L of L-glutamine, 2% of PS solution, and 10 ng/mL of brain-derived neurotrophic factor (BDNF). Viable cells were counted in a Neubauer cytometer, using the trypan blue exclusion test. The cells were seeded at a density of 25,000 per well in 96-well plates precoated with poly-L-lysine and will be cultured at 37° C. in an air (95%)-CO₂ (5%) incubator. On day 15 of culture, the compounds were dissolved in the culture medium. Primary cortical neurons were incubated with the compounds for 60 min before NMDA exposure. After the 60 min incubation with the compounds, NMDA was added to a final concentration of 30 μM diluted in control medium still in presence of the compounds for 1 hour. Then, live cells were incubated with MitoSOX™ Red (a marker of ROS generated specifically by the mitochondria) for 10 min at 37° C. The MitoSOX™ reagent is cell-penetrant and will become fluorescent once oxidized by superoxide. Then, cells were washed twice with warmed PBS cells fixed by cold solution of ethanol (95%) and acetic acid (5%) for 5 min at −20° C. Cell membranes will be permeabilized and non-specific binding sites will be blocked with a solution of PBS containing 0.1% of saponin and 1% FBS for 15 min at room temperature. Then, the cultures will be incubated with a mouse monoclonal antibody anti microtubule-associated protein 2 (MAP-2) at dilution of 1/400 in PBS containing 1% FBS and 0.1% of saponin. This antibody will be revealed with Alexa Fluor 488 goat anti-mouse IgG at the dilution 1/800 in PBS containing 1% FBS, 0.1% saponin, for 1 hour at room temperature.

Finally, the level of ROS in cortical neurons was quantified using ImageXpress®. For each condition, 20 pictures (representing the whole well area) were automatically taken using ImageXpress® (Molecular Devices) at 20× magnification using the same acquisition parameters (fluorescence reading at 488 nM for MAP-2 staining (green) and at 568 for ROS generated by mitochondria (red)). From images, analyses were directly and automatically performed by MetaXpress® (Molecular Devices) to quantify the ROS specifically in MAP-2-stained cortical neurons.

On day 15 of culture, 1 hour before the compound 2 application, Fluo 4 AM (4 μM) was incubated on the cells for 2 hours at 37° C. 2 hours after the Fluo 4 AM (4 μM) and 1 h after the compound 2 application, NMDA (30 μM) was applied to the cells. The level of intracellular Ca²⁺ (on total cells) was measured immediately after application of NMDA, and every 3 min for 1 hour, using Glomax apparatus.

### Results

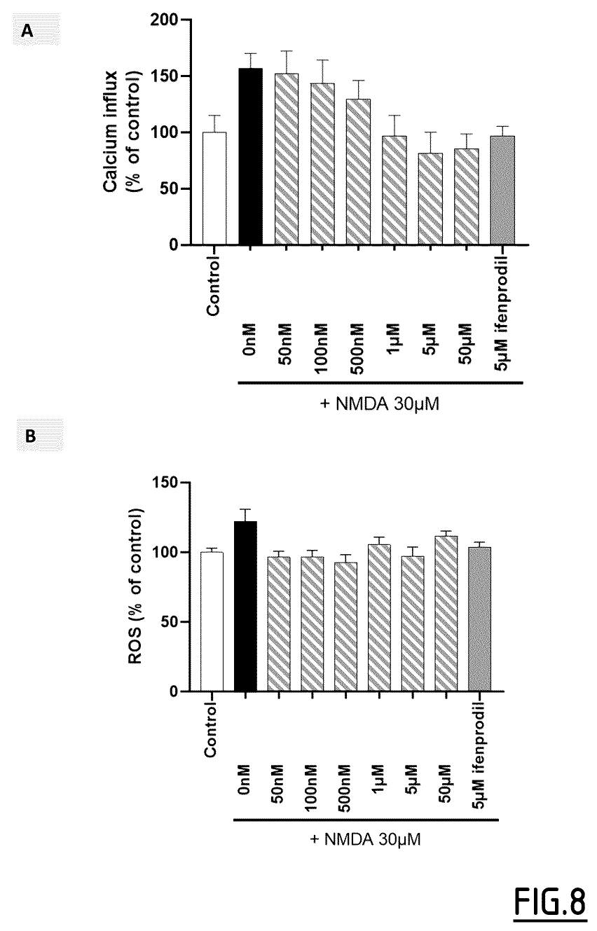

The compound 2 decreases the calcium flux inside WT rat cortical neurons stressed with 30 μM NMDA (FIG. 8A). Compound 2 displays is activity from nanomolar to micromolar concentrations. The compound 2 at nanomolar concentration is also able to decrease the ROS production in WT rat cortical neurons stressed with 30 μM NMDA (FIG. 8B). In this experimental setting, 1 μM concentration of compound 2 displays the same inhibitory effect on calcium influx or on ROS production than 5 μM ifenprodil.

1. A method of selectively inhibiting the subunit 2B (NR2B) of the N-methyl-D-aspartate (NMDA) receptor in a cell having NMDA receptor subunit 2B (NR2B)-containing NMDA receptors, the method comprising treating the cell with an effective amount of a compound of general formula (I): 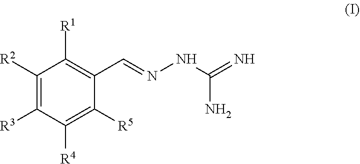
and the (Z) and/or (E) isomers thereof, or a tautomer thereof, or pharmaceutically acceptable salts thereof,
wherein: R1, R2, R3, R4, R5 are independently hydrogen, deuterium, halogen, haloalkyl, alkyl, alkoxy, hydroxyl, aryl, or aryloxy,
so as to effect a neuroprotective reduction in the effect of excitotoxic NMDA receptor activity.

2. The method according to claim 1 wherein the reduction in the effect of excitotoxic NMDA receptor activity in the cell is provided by a reduction of intracellular Ca²⁺ concentration.

3. The method according to claim 1 wherein the reduction in the effect of excitotoxic NMDA receptor activity in the cell is provided by a reduction of reactive oxygen species concentration.

4. A method of selectively inhibiting the subunit 2B (NR2B) of the N-methyl-D-aspartate (NMDA) receptor in a subject having NMDA receptor subunit 2B (NR2B)-containing NMDA receptors, the method comprising administering an effective amount of a compound of general formula (I): 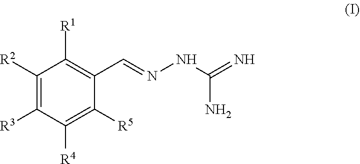
and the (Z) and/or (E) isomers thereof, or a tautomer thereof, or pharmaceutically acceptable salts thereof,
wherein: R1, R2, R3, R4, R5 are independently hydrogen, deuterium, halogen, haloalkyl, alkyl, alkoxy, hydroxyl, aryl, or aryloxy,
in said subject in need thereof.

5. A method for preventing or treating a disease, disorder, or medical condition caused by overactivation of the N-methyl-D-aspartate (NMDA) receptor containing a subunit 2B (NR2B) by selectively targeting the NR2B subunit of said NMDA receptor, wherein said method comprises administering to a subject in need thereof an effective amount of a compound of general formula (I): 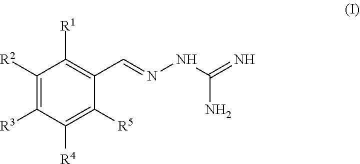
and the (Z) and/or (E) isomers thereof, or a tautomer thereof, or pharmaceutically acceptable salts thereof,
wherein: R1, R2, R3, R4, R5 are independently hydrogen, deuterium, halogen, haloalkyl, alkyl, alkoxy, hydroxyl, aryl, or aryloxy.

6. The method according to claim 5 wherein the disease, disorder, or medical condition is selected from the group consisting of:
(a) depression or a depressive disorder, a major depressive disorder, a treatment-resistant major depressive disorder, post-partum depression, bipolar depression;
(b) anxiety disorder, obsessive compulsive disorder, generalized anxiety disorder, agoraphobia with panic disorder, panic disorder, post-traumatic-stress disorder, social anxiety disorder;
(c) autism or autism spectrum disorder, Asperger's syndrome, or pervasive developmental disorder not otherwise specified (PDD-NOS);
(d) epilepsy, seizure disorder;
(e) migraine, chronic tension type headache (CTTH), migraine with allodynia, chronic headache;
(f) abnormal brain function, selected among Fragile X syndrome, tuberous sclerosis, Down's syndrome and other forms of mental retardation;
(g) withdrawal syndromes, e.g. alcohol, opioids or cocaine;
(h) pain, hyperalgesia, nociception, acute pain, chronic pain, or cancer-related pain;
(i) pain associated with excitotoxicity, preferably with glutamate excitotoxicity, and/or is associated with malfunctioning of glutamatergic neurotransmission;
(j) neuropathic pain;
(k) pseudobulbar affect (PBA).
(l) dyskinesia;
(m) amyotrophic lateral sclerosis (ALS) or bulbar-onset ALS;
(n) Charcot Marie Tooth disease (CMT);
(o) multiple sclerosis (MS);
(p) Parkinson's disease, atypical parkinsonian disorders (e.g. progressive supranuclear palsy);
(q) Alzheimer disease (AD), dementia, frontotemporal dementias (FTDs), progressive supranuclear palsy (PSP), corticobasal degeneration (CBD).
(r) Huntington's disease (HD);
(s) focal brain injuries from trauma, tumors or strokes;
(t) brain or spinal cord injury, peripheral nervous system injury, cerebral ischemia, head or neuronal trauma, neuronal hemorrhage, neuronal ischemia, reperfusion injury, neuronal injury;
(u) neuronal exposure to a toxic substance, methamphetamine-induced neurotoxicity;
(v) stroke, cardiogenic shock, coronary artery bypass graft (CABG) surgery associated neurological damage;
(w) Idiopathic pulmonary fibrosis (IPF) and chronic cough
and symptoms thereof.

7. The method according to claim 6 wherein the neuropathic pain is selected from the group consisting of: the peripheral neuropathic pain; central neuropathic pain; chronic neuropathic pain; refractory neuropathic pain; neuropathic pain associated with a metabolic dysfunction, including, for example, diabetes mellitus and pre-diabetes; neuropathic pain associated with diabetes mellitus; neuropathic pain associated with pre-diabetes; neuropathic pain associated with painful polyneuropathy; neuropathic pain associated with painful diabetic neuropathy, including, for example, diabetic peripheral neuropathy; neuropathic pain associated with painful diabetic polyneuropathy; neuropathic pain associated with post-herpetic neuralgia; neuropathic pain associated with trigeminal neuralgia; neuropathic pain associated with occipital neuralgia; neuropathic pain associated with painful radiculopathy, including, for example, lumbar and cervical painful radiculopathy; neuropathic pain associated with an infectious disease, including, for example, herpes zoster (shingles), HIV infection, Lyme disease, diphtheria, and leprosy; neuropathic pain associated with a liver or kidney disorder, including, for example, a chronic liver or kidney disorder, including, for example, liver disease, liver failure, kidney disease, and kidney failure; neuropathic pain associated with an immune or inflammatory disorder, including, for example, Guillain-Barre syndrome, rheumatoid arthritis, lupus, Sjörgren's syndrome, and coeliac disease; neuropathic pain associated with an inherited neuropathy or channelopathy, including, for example, inherited erythromelalgia, paroxysmal extreme pain disorder, and Charcot-Marie-Tooth disease (CMT); neuropathic pain associated with small fiber sensory neuropathy; neuropathic pain associated with a thyroid hormone disorder, including, for example, hypothyroidism; neuropathic pain associated with stroke; neuropathic pain associated with cancer, including, for example, lymphoma and multiple myeloma; neuropathic pain associated with chemotherapy, for example, cancer chemotherapy; neuropathic pain associated with peripheral nerve injury pain; neuropathic pain associated with nerve damage following traumatic injury; neuropathic pain associated with post-traumatic neuropathy; neuropathic pain associated with spinal cord injury, including, for example, spinal cord injury caused by trauma, for example, a road traffic accident; neuropathic pain associated with traumatic peripheral nerve injury; neuropathic pain associated with post-surgery neuropathy (e.g., post-surgery neuropathic pain); neuropathic pain following surgery, including, for example, neuropathic pain following nerve surgery, including, for example, spinal cord surgery; neuropathic pain associated with fibromyalgia; neuropathic pain associated with lower back pain; neuropathic pain associated with carpal tunnel syndrome; neuropathic pain associated with causalgia; neuropathic pain associated with reflex sympathetic dystrophy (RSD); neuropathic pain associated with Complex Regional Pain Syndrome (CRPS), including, for example, Type 1 and Type 2; neuropathic pain associated with amputation; neuropathic pain associated with a neurodegenerative disease, for example, Amyotrophic Lateral Sclerosis and Parkinson's disease; neuropathic pain associated with stroke, including, for example, central post-stroke pain; neuropathic pain associated with syringomyelia; neuropathic pain associated with a demyelinating disease, including, for example, multiple sclerosis, transverse myelitis, and neuromyelitis optica; or idiopathic neuropathic pain.

8. The method according to claim 6 which prevents, treats or alleviates pseudobulbar affect (PBA), or symptoms thereof, in a subject selected from the group consisting of patients having Parkinson's disease (PD), or atypical parkinsonian disorders (e.g. progressive supranuclear palsy), amyotrophic lateral sclerosis (ALS), bulbar-onset ALS, multiple sclerosis (MS), Alzheimer disease (AD), dementia, frontotemporal dementias (FTDs), progressive supranuclear palsy (PSP) and corticobasal degeneration (CBD), tumors, stroke.

9. The method according to claim 6 which prevents, treats or alleviates depression, or symptoms thereof, in a subject selected from the group consisting of patients having Parkinson's disease (PD), or atypical parkinsonian disorders (e.g. progressive supranuclear palsy), Alzheimer disease (AD), Huntington disease (HD), amyotrophic lateral sclerosis (ALS), bulbar-onset ALS, multiple sclerosis (MS), Charcot-Marie-Tooth disease (CMT).

10. The method according to claim 6 which prevents, treats or alleviates dyskinesia, or the symptoms thereof, in a subject selected from the group consisting of patients having Parkinson's disease (PD), atypical parkinsonian disorders (e.g. progressive supranuclear palsy), Huntington disease (HD).

11. The method according to claim 5 which does not simultaneously involve side effects chosen from psychotic effect, cognitive impairment and symptoms associated with schizophrenia.

12. The method according to claim 1 or 5 wherein in formula (I):
R1, R3 and R5 are independently chosen from H, Cl, F, Br and OH;
R2=R4=H.

13. The method according to claim 1 or 5 where the compound of formula (I) is chosen from the list consisting in:
2-(2,6-Dichlorobenzylidene)hydrazinecarboximidamide
2-(2-Chlorobenzylidene)hydrazinecarboximidamide
2-(2-Chloro-4-fluoro benzylidene)hydrazinecarboximidamide
2-(2-Chloro-6-fluorobenzylidene)hydrazinecarboximidamide
2-(2-Bromobenzylidene)hydrazinecarboximidamide
2-(2-Fluorobenzylidene)hydrazinecarboximidamide
2-(2,4-Difluorobenzylidene)hydrazinecarboximidamide
2-(2,6-Difluorobenzylidene)hydrazinecarboximidamide acetate salt
2-(2,4-Dichlorobenzylidene)hydrazinecarboximidamide acetate salt
2-(2,3-Dichlorobenzylidene)hydrazinecarboximidamide
2-(2,3,4-Trichlorobenzylidene)hydrazinecarboximidamide
2-(3,4,5-Trichlorobenzylidene)hydrazinecarboximidamide
2-(2,4,6-Trifluorobenzylidene)hydrazinecarboximidamide acetate salt
2-(2,4,5-Trifluorobenzylidene)hydrazinecarboximidamide
2-(2,6-Difluoro-4-chlorobenzylidene)hydrazinecarboximidamide
2-(2,4-Dichloro-3-fluorobenzylidene)hydrazinecarboximidamide
2-(2-Chloro-4,6-difluorobenzylidene)hydrazinecarboximidamide
2-(2-Chloro-4,5-difluorobenzylidene)hydrazinecarboximidamide
2-(2-Chloro-4-hydroxybenzylidene) hydrazinecarboximidamide
2-(2-Chloro-3-methylbenzylidene)hydrazinecarboximidamide
2-(2-Chloro-4-methylbenzylidene)hydrazinecarboximidamide
2-(2-Chloro-5-methylbenzylidene)hydrazinecarboximidamide
2-(2,4-Dichloro-6-fluorobenzylidene)hydrazinecarboximidamide
2-(2,6-Dichloro-4-fluorobenzylidene)hydrazinecarboximidamide
2-(2,3-Dichloro-4-fluorobenzylidene)hydrazinecarboximidamide
2-(2-Chloro-3, 5-difluorobenzylidene)hydrazinecarboximidamide
2-(3,4-Dichloro-6-fluorobenzylidene)hydrazinecarboximidamide
2-(3,5-Dichloro-4-fluorobenzylidene)hydrazinecarboximidamide
2-(2,4-Dichloro-5-fluorobenzylidene)hydrazinecarboximidamide
2-(2,3,5-Drichlorobenzylidene)hydrazinecarboximidamide
2-(3,4,5-Drifluorobenzylidene)hydrazinecarboximidamide
2-(2,3,4-Drifluorobenzylidene)hydrazinecarboximidamide
and the (Z) and/or (E) isomers thereof, or a tautomer thereof, or pharmaceutically acceptable salts thereof.

14. The method according to claim 1 or 5 wherein the compound of formula (I) is chosen from: 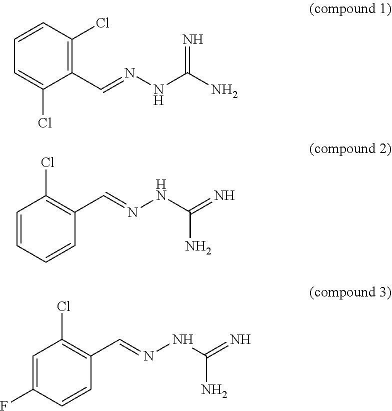
and the (Z) and/or (E) isomers thereof, or a tautomer thereof, or pharmaceutically acceptable salts thereof.

15. The method according to claim 1 or 5 wherein the compound of formula (I) is compound 2 of formula: 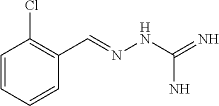 [SMILES: N=C(N)N/N=C/c1ccccc1Cl]
and the (Z) and/or (E) isomers thereof, or a tautomer thereof, or pharmaceutically acceptable salts thereof.

16. The method according to claim 1 or 5 wherein the compound of formula (I) is (Z) isomer of compound 1:

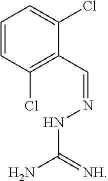 [SMILES: N=C(N)N/N=C\c1c(Cl)cccc1Cl]

17. The method according to claim 1 or 5 wherein the subject is a human.

18. A compound selected from the group consisting in:
2-(2,4,6-Trifluorobenzylidene)hydrazinecarboximidamide acetate salt
2-(2,6-Difluoro-4-chlorobenzylidene)hydrazinecarboximidamide
2-(2-Chloro-4,6-difluorobenzylidene)hydrazinecarboximidamide
2-(2-Chloro-4-hydroxybenzylidene)hydrazinecarboximidamide
2-(2,4-Dichloro-6-fluorobenzylidene)hydrazinecarboximidamide
2-(2,6-Dichloro-4-fluorobenzylidene)hydrazinecarboximidamide
2-(2-Dichloro-3, 5-difluorobenzylidene)hydrazinecarboximidamide
and the (Z) and/or (E) isomers thereof, or a tautomer thereof, or pharmaceutically acceptable salts thereof.
